(19) United States
(12) Patent Application Publication
(10) Pub. No.: US 2015/0051242 A1
(43) Pub. Date: Feb. 19, 2015

(54) RAFAMYCIN ANALOGS AND METHODS FOR MAKING SAME

(71) Applicant: HANGZHOU ZYLOX PHARMA CO., LTD, Hangzhou, Zhejiang (CN)

(72) Inventor: Jonathon Z. Zhao, Hangzhou (CN)

(73) Assignee: ZHEJIANG ZYLOX MEDICAL DEVICE CO., LTD., Hangzhou, Zhejiang (CN)

(21) Appl. No.: 14/383,477

(22) PCT Filed: Nov. 30, 2012
(86) PCT No.: PCT/CN2012/085631

§ 371 Date: Sep. 5, 2014

(30) Foreign Application Priority Data
Oct. 26, 2012 (US) 61718964

(51) Int. Cl. C07D 498/18

(52) U.S. Cl. CPC C07D 498/18
USPC 514/292; 540/456

(57) ABSTRACT

A semi-synthetic rapamycin analog with a triazole moiety or a pharmaceutically acceptable salt or prodrug thereof, is a broad-spectrum cytostatic agent and a mTOR inhibitor, and is useful in the treatment of various cancers, or tumors in organs such as kidney, liver, breast, head and neck, lung, prostate, and restenosis in coronary arteries, peripheral arteries, and arteries in the brain, immune and autoimmune diseases. Also disclosed are fungal growth-, restenosis-, post-transplant tissue rejection- and immune- and autoimmune disease-inhibiting compositions and a method of inhibiting cancer, fungal growth, restenosois, post-transplant tissue rejection, and immune and autoimmune disease in a mammal. One particular preferred application of such triazole-moiety containing rapamycin analog is in treating renal carcinoma, lung cancer, colon cancer, and breast cancers wherein potency of the drug, its half-life, tissue distribution properties, and its pharmacokinetic properties including bioavailability through oral and intravenous routes are essential to the clinical outcomes.

### BACKGROUND OF THE INVENTION

The compound cyclosporine (cyclosporin A) has found wide use since its introduction in the fields of organ transplantation and immunomodulation, and has brought about a significant increase in the success rate for transplantation procedures. Recently, several classes of macrocyclic compounds having potent immunomodulatory activity have been discovered. Okuhara et al., in European Patent Application No. 184, 162, published Jun. 11, 1986, discloses a number of macrocyclic compounds isolated from the genus Streptomyces, including the immunosuppressant FK-506, a 23-membered macrocyclic lactone, which was isolated from a strain of S. tsukubaensis.

Other related natural products, such as FR-900520 and FR-900523, which differ from FK-506 in their alkyl substituent at C-21, have been isolated from S. hygroscopicus yakushimnaensis. Another analog, FR-900525, produced by S. tsukubaensis, differs from FK-506 in the replacement of a pipecolic acid moiety with a proline group. Unsatisfactory side-effects associated with cyclosporine and FK-506, such as nephrotoxicity, have led to a continued search for immunosuppressant compounds having improved efficacy and safety, including an immunosupressive agent which is effective topically, but ineffective systemically (U.S. Pat. No. 5,457,111).

Rapamycin, as illustrated below, is a macrocyclic triene antibiotic produced by Streptomyces hygroscopicus, which was found to have antifungal activity, particularly against Candida albicans, both in vitro and in vivo (U.S. Pat. No. 3,929,992 and U.S. Pat. No. 3,993,749).

Rapamycin alone (U.S. Pat. No. 4,885,171) or in combination with picibanil (U.S. Pat. No. 4,401,653) has been shown to have antitumor activity. In 1977, rapamycin was also shown to be effective as an immunosuppressant in the experimental allergic encephalomyelitis model, a model for multiple sclerosis: in the adjuvant arthritis model, a model for rheumatoid arthritis: and was shown to effectively inhibit the formation of IgE-like antibodies.

The immunosuppressive effects of rapamycin have also been disclosed in FASEB in 1989, as has its ability to prolong survival time of organ grafts in histoincompatible rodents. These and other biological effects of rapamycin are reviewed in Transplantation Reviews, 1992, 6, 39-87. Mono-ester and di-ester derivatives of rapamycin (esterification at positions 31 and 42) have been shown to be useful as antifungal agents (U.S. Pat. No. 4,316,885) and as water soluble prodrugs ofrapamycin (U.S. Pat. No. 4,650,803).

Mono-ester and di-ester derivatives of rapamycin (esterification at positions 31 and 42) have been shown to be useful as antifungal agents (U.S. Pat. No. 4,316,885) and as water soluble prodrugs ofrapamycin (U.S. Pat. No. 4,650,803).

Numerous chemical modifications of rapamycin have been attempted. These include the preparation of mono- and di-ester derivatives of rapamycin (WO 92/05179). 27-oximes of rapamycin (EPO 467606): 42-oxo analog of rapamycin (U.S. Pat. No. 5,023,262); bicyclic rapamycins (U.S. Pat. No. 5,120,725): rapamycin dimers (U.S. Pat. No. 5,120,727): silyl ethers of rapamycin (U.S. Pat. No. 5,120,842); and arylsulfonates and sulfamates (U.S. Pat. No. 5,177,203). Rapamycin was recently synthesized in its naturally occurring enantiomeric form (K. C. Nicolaou et al., J. Am. Chem. Soc, 1993, 115, 4419-4420: S. L. Schreiber, J. Am. Chem. Soc. 1993, 115, 7906-7907; S. J. Danishefsky, J. Am. Chem. Soc. 1993, 115, 9345-9346). One recent example of a rapamycin analog is a tetrazole containing rapamycin analog (U.S. Pat. No. 6,015,815). The tetrazole heterocyclic ring is used to replace the hydroxyl group to effect the analog.

Although some of these modified compounds exhibit immunosuppressive activity, anti-restenotic activities in suppressing the migration and growth of vascular smooth muscles, especially when used in a stent coating, the need remains for rapamycin analogs which possess potentially enhanced efficacy against broad spectrum of cancers such as renal cell carcinoma, breast cancers, head and neck cancers, and potentially better lipophilicity. longer half live in the blood or in local tissues, or resistance to oxidative forces and better stability in a formulation. One way to achieve these goals is through introduction of a triazole moiety to the side chain of a rapamycin which may impart a better lipophilicity, better stability, better bioavailability, better tissue and cellular uptake, better efficacy compared to the known and existing modified rapamycin analogs or derivatives. The efficacy of the modified rapamycin may also have better potency against a variety of cancers, and potentially reduced toxicities.

### SUMMARY OF THE INVENTION

Accordingly, one object of the present invention is to provide novel semi-synthetic rapamycin analogs which possess a desired triazole moiety attached to either or both to 31C-, and or 42C-position of a rapamycin molecule.

In accordance with one aspect, the present invention is directed to compounds represented by the structural formula illustrated below.

In accordance with one aspect, the present invention is directed to compounds represented by the structural formula illustrated below.

In accordance with yet another aspect, a compound of the present invention may contain two such substitutes at both the 42C and 31C-positions of a rapamycin.

The triazole moiety of the present invention may be introduced via a variety of reaction schemes, the typical ones are illustrated below:

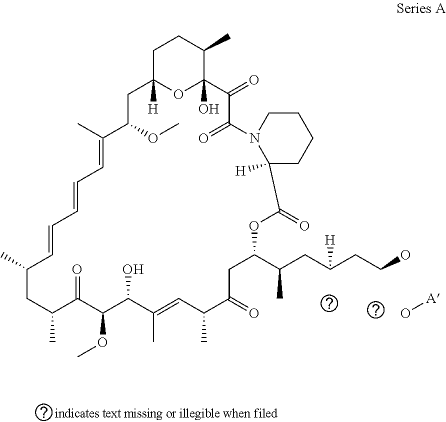

wherein A′ is one of the following structures:

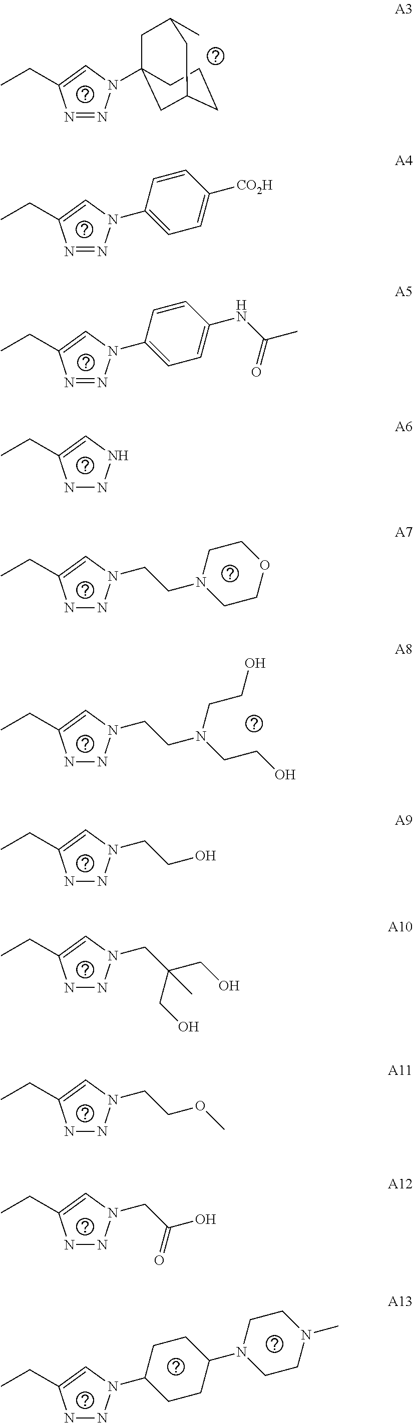

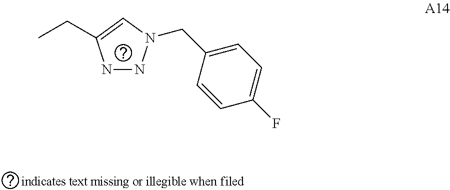

### Series B:

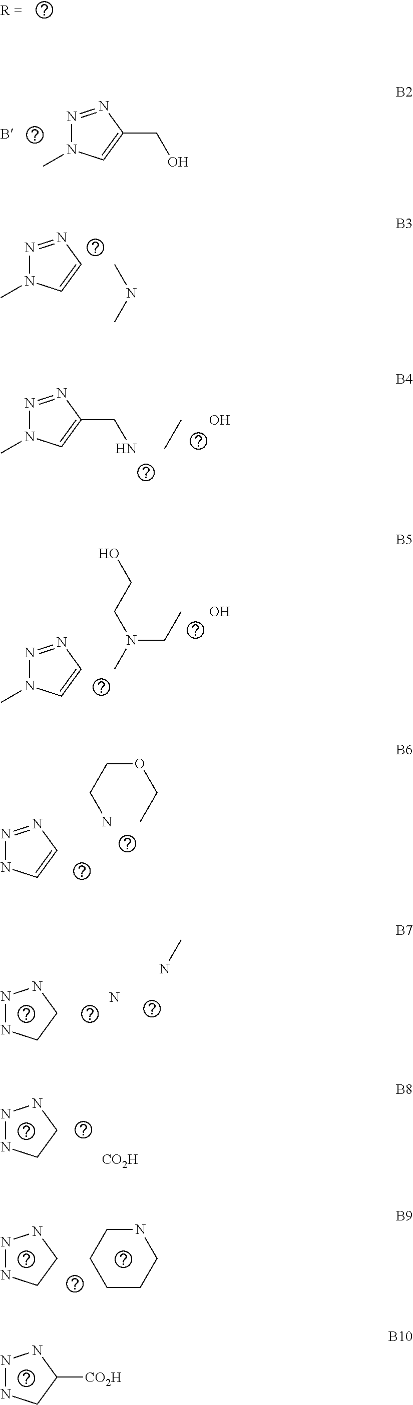

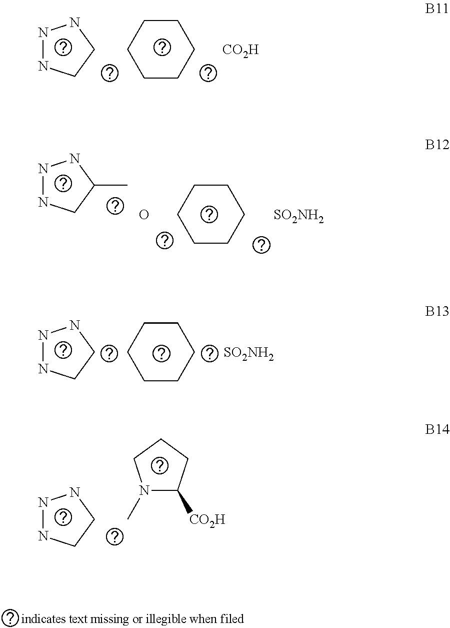

Another object of the present invention is to provide a synthetic processes for the preparation of such compounds from starting materials obtained by fermentation, as well as chemical intermediates useful in such synthetic processes.

A further object of the present invention is to provide pharmaceutical compositions containing, as an active ingredient, at least one of the above compounds.

Yet another object of the present invention is to provide a method of treating a variety of disease states, including restenosis, post-transplant tissue rejection, immune and autoimmune dysfunction, fungal growth, and cancer.

In addition, the compounds of the present invention may be employed as an oral tablet, oral solid or oral liquid, oral immediate or sustained release dosage, intravenous injection dosages, parenteral dosages, cream or solutions by formulation with pharmaceutically acceptable vehicles.

Also within the scope of this invention includes pharmaceutical compositions for immediate release or sustained release of its active ingredient, each comprising a compound of this invention and pharmaceutically acceptable excepient.

Still further related to this invention are medical devices, each comprising a compound of this invention. Examples of the medical deices include drug-eluting coronary or peripheral, esophageal, urinary, ovary, or neurovascular stent.

### BRIEF DESCRIPTION OF THE DRAWINGS

FIG. 1 Graph for Renal cell carcinoma tumor cell inhibition studies;

FIG. 2 Graph for Renal cell carcinoma tumor cell inhibition studies;

FIG. 3 Graph for Lung Cancer A549 cell inhibition studies;

FIG. 4 Graph for Lung Cancer A549 cell inhibition studies;

FIG. 5 Graph for Lung Cancer A549 cell inhibition studies;

FIG. 6 Graph for Melanoma SK-MEL-28 cell inhibition studies;

FIG. 7 Graph for Melanoma SK-MEL-28 cell inhibition studies;

FIG. 8 Graph for Melanoma SK-MEL-28 cell inhibition studies;

FIG. 9 Graph for Epidermal cancer A431 tumor cell model;

FIG. 10 Graph for Epidermal cancer A431 tumor cell model;

FIG. 11 Graph for Epidermal cancer A431 tumor cell model;

FIG. 12 Graph for Glioblastoma U87 MG Tumor model studies;

FIG. 13 Graph for Glioblastoma U87 MG Tumor model studies;

FIG. 14 Graph for Glioblastoma U87 MG Tumor model studies;

FIG. 15 Graph for Human colorectal tumor HCT 116 model studies;

FIG. 16 Graph for Human colorectal tumor HCT 116 model studies;

FIG. 17 Graph for Human colorectal tumor HCT 116 model studies;

FIG. 18 Graph for Breast cancer MDA-MB-231 tumor model;

FIG. 19 Graph for Breast cancer MDA-MB-231 tumor model;

FIG. 20 Graph for Breast cancer MDA-MB-231 tumor model;

FIG. 21 Graph for Breast cancer MCF-7 tumor model;

FIG. 22 Graph for Breast cancer MCF-7 tumor model;

FIG. 23 Graph for Breast cancer MCF-7 tumor model;

FIG. 24 Graph for Prostate cancer PC-3 tumor studies;

FIG. 25 Graph for Prostate cancer PC-3 tumor studies;

FIG. 26 Graph for Prostate cancer PC-3 tumor studies;

FIG. 27 Efficacy of rapamycin analog of the present invention in treating HCT 116.

### DETAILED DESCRIPTION OF THE PREFERRED EMBODIMENTS

### Definition of Terms

The term “prodrug,” as used herein, refers to compounds which are rapidly transformed in vivo to the parent compound of the above formula, for example, by hydrolysis in blood. A thorough discussion is provided in T. Higuchi and V. Stella, “Pro-drugs as Novel Delivery Systems,” Vol. 14 of the A.C.S. Symposium Series, and in Edward B. Roche, ed., “Bioreversible Carriers in Drug Design.” American Pharmaceutical Association and Pergamon Press, 1987, both of which are hereby incorporated by reference.

The term “pharmaceutically acceptable prodrugs,” as used herein, refers to those prodrugs of the compounds of the present invention which are, within the scope of sound medical judgment, suitable for use in contact with the tissues of humans and lower mammals without undue toxicity, irritation, and allergic response, are commensurate with a reasonable benefit/risk ratio, and are effective for their intended use, as well as the zwitterionic forms, where possible, of the compounds of the present invention. Particularly preferred pharmaceutically acceptable prodrugs of the present invention are prodrug esters of the C-31 hydroxyl group of compounds of the present invention.

The term “prodrug esters,” as used herein, refers to any of several ester-forming groups that are hydrolyzed under physiological conditions. Examples of prodrug ester groups include acetyl, ethanoyl, pivaloyl, pivaloyloxymethyl, acetoxymethyl, phthalidyl, methoxymethyl, indanyl, and the like, as well as ester groups derived from the coupling of naturally or unnaturally-occurring amino acids to the C-31 hydroxyl group of compounds of the present invention.

The term “isomer” as used herein, refers to a compound having the identical chemical formula but different structural or optical configurations.

The term “epimer” as used herein, refers to a compound having the identical chemical formula but a different optical configuration at a particular position. In the case of a rapamycin, a 42-Epi rapamycin refers to the compound that has the opposite optical rotation compared to the rapamycin obtained by a fermentation process.

The term “15-isomer” as used herein, refers to the analog of rapamycin that contains a 7-member ring at the 15-position as opposed to a regular rapamycin obtained from a fermentation process which contains a six-member ring. This kind of conversion is also called “tautomerization”. The 15-isomer” as used herein, may also be referred to as a 15 tautomer of a rapamycin.

### Preparation of Compounds

The compounds and processes of the present invention will be better understood in connection with the following synthetic schemes which illustrate the methods by which the compounds of the present invention may be prepared.

The compounds of the present invention may be prepared by a variety of synthetic routes. Most of the common conjugation reactions of rapamycin at 42- and/or 31-hydroxyl positions are found in the rapamycin patents mentioned above, the contents of which are incorporated herein by reference in their entireties.

### EXAMPLES

Synthesis of Rapamycin Derivatives. The parent rapamycin structure is shown below.

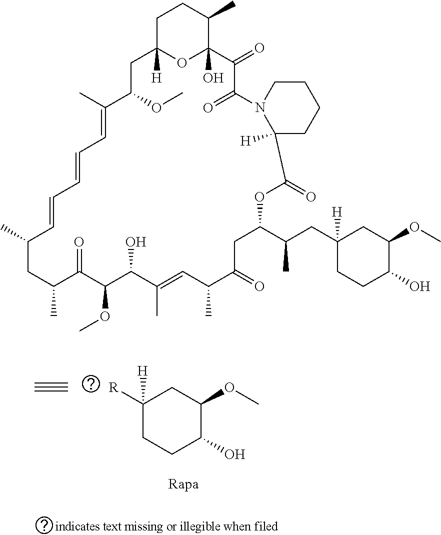

The synthetic scheme of series A of rapamycin analogs of the present invention is shown below:

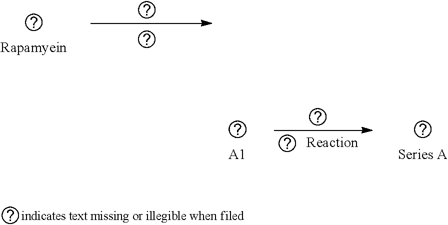

Shown below are additional rapamycin analogs of this invention that were synthesized similarly:

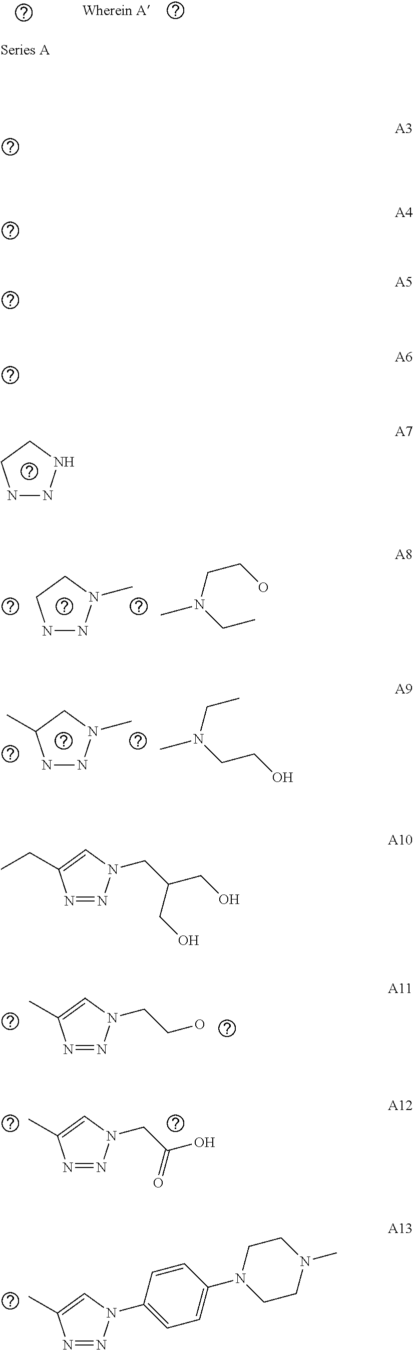

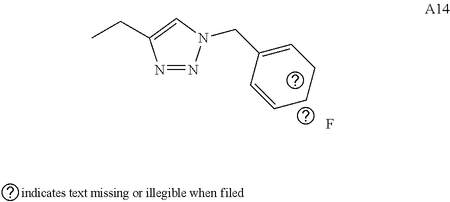

### Example 1: Synthesis of Compound A1

To a stirred solution of Rapamycin (3 g, 3.2 mmol) and Cs₂CO₃ (3.2 g, 9.6 mmol) in dried PMF (90 mL) was added Nal (1.5 g, 9.6 mmol) and 3-bromoprop-1-yne (1.2 g, 9.6 mmol). The reaction mixture was stirred at rt for 30 hours. Upon the completion of reaction, 300 mL water was added in and extracted with ethyl acetate (200 mL×3). The combined organic layer was washed by brine (300 mL) and dried over anhydrous Na₂SO₄. After concentration, the residue was purified with silica gel chromatography (50% to 100% of ethyl acetate in petroleum ether as eluent) to give the compound A1 (2.1 g, 68%) as a light green oil.

LCM (m/z) ES-950 (M−1)⁻.

### Example 2: Synthesis of Compound A3

To a solution of 40-O-(prop-2-ynylox) rapamycin A1 (200 mg, 0.2 mmol) and 1-azido-Admantane (100 mg, 0.6 mmol) in anhydrous THF (9 mL) was added DIPEA (100 μL, 0.6 mmol) and CuI (20 mg, 0.1 mmol) under N₂. The solution was stirred at rt overnight. Then, 20 mL water was added and extracted with ethyl acetate (20 mL×3). The combined organic layer was washed by brine and dried over anlndrous Na₂SO₄. After concentration, the residue was purified with silica gel chromatography (25% to 50% of ethyl acetate in petroleum ether as eluet) to give white solid which was further purified by prep-HPLC to give Compound A3 (26 mg, 10%) as a white solid. ¹H NMR (300 MHz, CDCl₃) δ7.71 (s, 1H), 6.74 (m, 1H), 6.39-6.02 (m, 5H), 5.62-5.36 (m, 5H): LCMS (m/z) ES-1128 (M−1)⁻.

### Example 3: Synthesis of Compound A4

To a solution of 40-O-(prop-2-ynyloxy) rapamycin A1 (200 mg, 0.2 mmol) and 4-azidobenzoic acid (100 mg, 0.6 mmol) in anhydrous THF (9 mL) was added DIPEA (100 μL, 0.6 mmol) and CuI (20 mg, 0.1 mmol) under N₂. The solution was stirred at rt for 3 hours. Then, 20 mL water was added and extracted with ethyl acetate (20 mL×3). The combined organic layer was washed by brine and dried over anhydrous Na₂SO₄. After concentration, the residue was purified with silica gel chromatography (5% to 10% of methanol in dichloromethane as eluent) to give white solid which was further purified by PREP-HPLC to give Compound A4 (29 mg, 12%) as a white solid. ¹H NMR (300 MHz, CDCl₃) δ8.27 (m, 1H), 7.90 (m, 1H), 7.73 (m, 1H), 7.56 (m, 1H), 6.74 (m, 1H), 6.55-6.00 (m, 5H), 5.60-5.36 (m, 5H); ECMS (m/z) ES-1114(M−1)⁻.

### Example 4: Synthesis of Compound A5

### Preparation of Intermediate 2

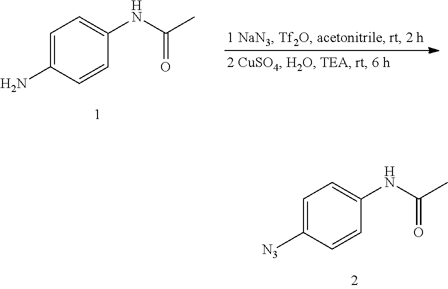

To a stirred suspension of NaN₃ (2.0 g, 30.8 mmol) in acetonitrile (20 mL) was added Tf₂O (7.3 g, 25.8 mmol) via syringe slowly at 0° C. The mixture was stirred for another 2 h at this temperature. The insoluble solids were removed through filtration. At 0° C., the filtrate was added dropvvise into the mixture of Compound 1 (2.0 g, 13 mmol). CuSO₄ (160 mg, 1 mmol), H₂O (6 mL) and Et3N (3.6 mL, 25.8 mmol). The reaction mixture was stirred for 6 h at room temperature. The mixture was diluted with EtOAc and washed with brine. The organic layer was dried over Na₂SO₄ and evaporated to yield brown solid which was purified with silica gel chromatography (30% to 50% of EtOAc in petroleum ether as eluent) to give Intermediate 2 (1.1 g, 48%) as a white solid. ¹H NMR (300 MHz, CDCl₃) δ9.93 (s, 1H), 7.63 (m, 2H), 7.03 (m, 2H), 2.02 (s, 3H): LCMS (m/z) ES+ 177 (M+1)⁺.

To a solution of 40-O-(prop-2-ynyloxy) rapamycin A1 (200 mg, 0.2 mmol) and N-(4-azido-phenyl) acetamide. Intermediate 2 (100 mg, 0.6 mmol) in anhydrous THF (9 mL) was added DIPEA (100 μL, 0.6 mmol) and CuI (20 mg, 0.1 mmol) under N₂. The solution was stirred at rt for 4 hours. Then, 20 mL water was added and the mixture was extracted with EtOAc (20 mL×3). The combined organic layer was washed by brine and dried over anhydrous Na₂SO₄. After concentration, the residue was purified with silica gel chromatography (30% to 100% of EtOAc in petroleum as eluent) to give white solid which was further purified by prep-HPLC to give Compound A5 (56 mg, 25%) as a white solid. ¹H NMR (300 MHz, CDCl₃) δ8.13 (m, 1H), 7.73 (m, 4H), 6.74 (m, 1H), 6.49-6.00 (m, 5H), 5.65-5.37 (m, 5H): LCMS (m/z) ES− 127 (M−1)⁺.

### Example 5: Synthesis of Compound A6

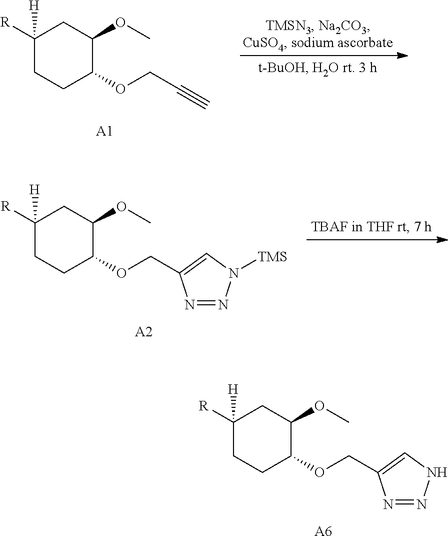

To a solution of 40-O-(prop-2-ynloxy) rapamycin A1 (200 mg, 0.2 mmol) and TMS-N₃ (100 mg, 0.9 mmol) in t-BuOH (6 mL) and H₂O (6 mL) was added Na₂CO₃ (100 mg, 1 mmol), CuSO₄ (20 mg, 0.13 mmol) and sodium ascorbate (40 mg, 0.2 mmol) under N₂. The solution was stirred at rt for 3 hours. Then, 20 mL water was added and extracted with EtOAc (20 mL×3). The combined organic layer was washed by brine and dried over anhydrous Na₂SO₄. After concentration, the residue was purified with silica gel chromatography (25% of EtOAc in petroleum ether as eluent) to give compound A2 (189 mg, 82%) as a white solid which was dissolved in TBAF in THF (10 mL) at 0° C. and stirred at rt for 7 hours. Then the reaction mixture was partitioned between EtOAc and water. The aqueous phase was extracted with EtOAc (25 mL×3). The combined organic layer was washed with brine and dried over anhydrous Na₂SO₄. After concentration, the residue was purified with silica gel chromatography (5% to 10% of methanol in dichloromethanc as eluent) to give white solid which was further purified by prep-HPLC to give compound A6 (38 mg, 24%) as a white solid, ¹H NMR (300 MHz, CDCl₃) δ7.75-7.55 (m, 1H), 6.76 (m, 1H), 6.49-6.08 (m, 5H), 5.53-5.35 (m, 3H); LCMS (m/z) ES− 1012 (M−1+18)⁺.

### Example 6: Synthesis of Compound A7

Preparation of intermediates 5 and 6:

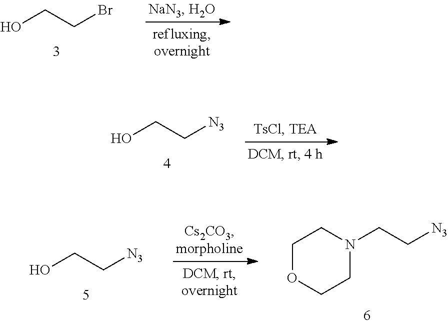

To a solution of 3 (5 g, 41 mmol) in 100 mL of water was added NaN₃ (5 g, 83 mmol) and was relluxed overnight. Then 100 mL DCM was added in after reaction mixture was cooled to rt. The organic phase separated was dried over Na₂SO₄, filtered. To the solution was added Et₃N (5.05 g, 50 mmol) and TsCl (9.55 g, 50 mmol) at 0 deg. The reaction mixture was stirred at rt for 4 hours. 100 mL water was added. The organic phase was separated and dried over Na₂SO₄. Filtration and concentration in vacuo gave the crude product. Purification by column chromatography (10% of EtOAc in petroleum ether as eluent) gave intermediate 5 (5.7 g, 58%) as a colorless oil. ¹H NMR (300 MHz, CDCl₃) δ7.81 (d, 2H), 7.39 (d, 2H), 4.14 (m, 2H), 3.48 (m, 2H), 2.43 (s, 3H): LCMS (m/z) ES+242 (M+1)⁺.

To a solution of intermediate 5 (1 g, 4.1 mmol) and Cs₂CO₃ (2.8 g, 8.2 mmol) in 30 mL of anhydrous DMF was added morpholine (0.71 g, 8.2 mmol) at 0° C. Then it was stirred at rt overnight. The reaction mixture was partitioned between 50 mL of EtOAc and 60 mL of water. The organic phase was dried over Na₂SO₄. Filtration and concentration in vacuo gave the crude product. Purification by column chromatography (50% of EtOAc in petroleum ether as eluent) gave intermediate 6 (0.4 g, 72%) as a colorless oil. LCMS (m/z) ES+ 157 (M+1)⁺.

To a solution of 40-O-(prop-2-ynyloxy) rapamycin A1 (200 mg, 0.2 mmol) and 4-(2-azido-ethyl)morpholine. Intermediate 6 (100 mg, 0.6 mmol) in anhydrous THF (9 mL) was added DIPEA (100 μL, 0.6 mmol) and CuI (20 mg, 0.1 mmol) under N₂. The solution was stirred at rt for 3 hours. Then, 20 mL water was added and extracted with EtOAc (20 mL×3). The combined organic layer was washed by brine and dried over anhydrous Na₂SO₄. After concentration, the residue was purified with silica gel chromatography (30% of EtOAc in petroleum ether as eluent) to give white solid which was further purified by prep-HPLC to give compound A7 (45 mg, 20%) as a white solid. ¹H NMR (300 MHz, CDCl₃) δ7.89 (m, 1H), 6.72 (m, 1H), 6.44-6.05 (m, 5H), 5.60-5.37 (m, 5H): LCMS (m/z) ES− 107 (M−1)⁻.

### Example 7: Synthesis of Compound A9

To a solution of 40-O-(prop-2-ynyloxy) rapamycin A1 (200 mg, 0.2 mmol) and 2-azidoethanol (100 mg, 1.2 mmol) in anhydrous THF (9 mL) was added DIPEA (100 μL, 0.6 mmol) and CuI (20 mg, 0.1 mmol) under N₂. The solution was stirred at rt overnight. Then, 20 mL water was added and extracted with EtOAc (20 mL×3). The combined organic layer was washed by brine and dried over anhydrous Na₂SO₄. After concentration, the residue was purified with silica gel chromatography (5% to 10% of methanol in dichloromethane as eluent) to give white solid which was further purified by prep-HPLC to give compound A9 (26 mg, 11%) as a white solid. ¹H NMR (300 MHz, CDCl₃) δ8.03-7.78 (m, 1H), 6.70 (m, 1H), 6.46-6.00 (m, 5H), 5.61-5.39 (m, 5H): LCMS (m/) ES− 1038 (M−1)⁻.

### Example 8: Synthesis of Compound A10

### Preparation of Intermediate 8

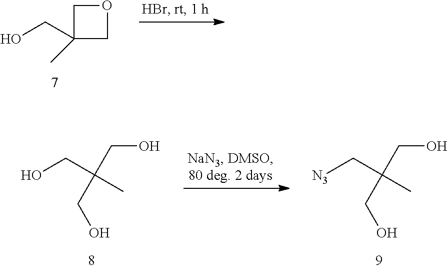

A solution of compound 7 (1.3 g, 12.7 mmol) in 40% HBr (10 mL) was stirred at rt for 1 hour. Then, 20 mL water was added and extracted with EtOAc (20 mL×3). The combined organic layer was washed by brine, dried over anhydrous Na₂SO₄. After concentration, the residue was purified with silica gel chromatography (50% of EtOAc in petroleum ether as eluent) to give intermediate 8 (0.7 g, 31%) as a white solid.

LCMS (m/z) ES+ 183 (M+1)⁺.

### Preparation of Intermediate 9

A solution of intermediate 8 (0.7 g, 3.9 mmol) and NaN₃ (1.13 g, 15 mmol) in DMSO (16 mL) was stirred at 80 deg for 2 days. Then, 20 mL water was added and extracted with EtOAc (20 mL×3). The combined organic layer was washed by brine, dried over anhydrous Na₂SO₄. After concentration, the residue was purified with silica gel chromatography (50% to 100% of EtOAc in petroleum ether as eluent) to give intermediate 9 (0.25 g, 45%) as a white solid.

LCMS (m/z) ES+ 146 (M+1)⁺.

### Preparation of Compound A 10

To a solution of 40-O-(prop-2-ynyloxy) rapamycin A1 (200 mg, 0.2 mmol) and 2-(azidomethyl) 2-methylpropane-1,3-diol Intermediate 9 (100 mg, 0.7 mmol) in t-BuOH (6 mL) and H₂O (6 mL) was added Na₂CO₃ (100 mg, 1 mmol), CuSO₄ (20 mg, 0.13 mmol) and sodium ascorbate (40 mg, 0.2 mmol) under N₂. The solution was stirred at rt for 6 hours. Then, 20 mL water was added and extracted with EtOAc (20 mL×3). The combined organic layer was washed by brine and dried over anhydrous Na₂SO₄. After concentration, the residue was purified with silica gel chromatography (5% to 10% of methanol in dichloromethane as eluent) to give white solid which was further purified b prep-HPLC to give compound A 10 (15 mg, 7%) as a white solid. ¹H NMR (300 MHz, CDCl₃) δ7.76 (m, 1H), 6.69 (m, 1H), 6.55-6.00 (m, 5H), 5.63-5.33 (m, 5H), LCMS (m/z) ES− 1096 (M−1)⁻.

### Example 9: Synthesis of Compound A12

### Preparation of Intermediate 11

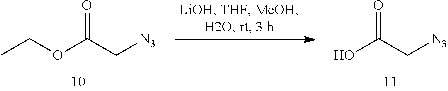

To a mixture of compound 10 (1 g, 7.8 mmol) in of MeOH/THF (10 mL/10 mL) was added a solution of LiOH (0.9 g, 39 mmol) in 10 mL of water. The resulting solution is stirred at room temperature for 3 hours. The mixture was acidified by 2N HCl to PH 4, and extracted with EtOAc (25 mL×2). The combine organic layer was concentrated under vacuum to give intermediate 11 (0.7 g, 91%) as a colorless oil. ¹NMR (300 MHz, CDCl₃) δ2.34 (s, 2H); LCMS (m/z) ES+ 102 (M+1)⁺.

### Preparation of Compound A 12

To a solution of 40-O-(prop-2-ynyloxy) rapamycin A1 (200 mg, 0.2 mmol) and 2-azidoacetic acid Intermediate 11 (100 mg, 1 mmol) in t-BuOH (6 mL) and H₂O (6 mL) was added Na₂CO₃ (100 mg, 1 mmol), CuSO₄ (20 mg, 0.13 mmol) and sodium ascorbate (40 mg, 0.2 mmol) under N₂. The solution was stirred at rt for 2 hours. Then, 20 mL water was added and extracted with EtOAc (20 mL×3). The combined organic layer was washed by brine and dried over anhydrous Na₂SO₄. After concentration, the residue was purified with silica gel chromatography (5% to 20% of methanol in dichloromethane as eluent) to give white solid which was further purified by prep-HPLC to give compound A12 (15 mg, 7%) as a white solid. ¹H NMR (300 MHz, CDCl₃) δ7.89 (m, 1H), 6.72 (m, 1H), 6.49-6.08 (m, 5H), 5.60-5.35 (m, 5H): LCMS (m/z) ES− 1052 (M−1)⁻.

### Example 10: Synthesis of Compound A13

### Preparation of Intermediate 13

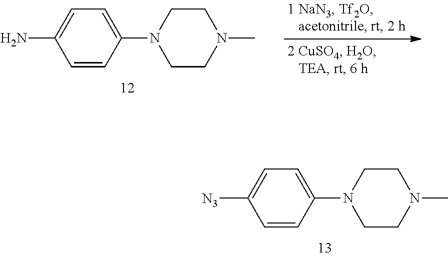 [SMILES: CN1CCN(c2ccc(N)cc2)CC1 ; CN1CCN(c2ccc(N=[N+]=[N-])cc2)CC1]

To a stirred suspension of NaN₃ (2.0 g, 30.8 mmol) in acetonitrilc (20 mL) was added Tf₂O (7.3 g, 25.8 mmol) by sringe slowly at 0 deg. The mixture was stirred for another 2 h at this temperature. The insoluble solids were removed through filtration. At 0 deg, the filtrate was added dropwise into the mixture of compound 12 (2.0 g, 10 mmol), CuSO₄ (160 mg, 1 mmol), H₂O (6 mL) and Et3N (3.6 mL, 25.8 mmol). The reaction mixture was stirred for 6 h at room temperature. The mixture was diluted with EtOAc and washed with brine. The organic layer was dried over Na₂SO₄ and evaporated to yield brown solid which was purified with silica gel chromatography (30% to 50% of EtOAc in petroleum ether as eluent) to give brown solid which was further purified by prep-HPLC to give intermediate 13 (0.4 g, 17%) as a brown solid. ¹H NMR (300 MHz, CDCl₃) δ6.04 (m, 4H), 3.19 (m, 4H), 2.60 (m, 4H), 2.36 (s, 3H): LCMS (m/z) ES+ 218 (M+1)⁺.

### Preparation of Compound A 13

To a solution of 40-O-(prop-2-ynyloxy) rapamycin A1 (200 mg, 0.2 mmol) and 1-(4-azido-phenyl)-4-methylpiperazine Intermediate 13 (100 mg, 0.5 mmol) in t-BuOH (6 mL) and H₂O (6 mL) was added Na₂CO₃ (100 mg, 1 mmol), CuSO₄ (20 mg, 0.13 mmol) and sodium ascorbate (40 mg, 0.2 mmol) under N₂. The solution was stirred at rt for 3 hours. Then, 20 mL water was added and extracted with EtOAc (20 mL×3). The combined organic layer was washed by brine and dried over anhydrous Na₂SO₄. After concentration, the residue was purified with silica gel chromatography (5% to 20% of methanol in dichloromethane as eluent) to give white solid which was further purified by prep-HPLC to give compound A13 (33 mg, 14%) as a white solid. ¹H NMR (300 MHz, CDCl₃) δ8.11 (m, 1H), 7.71 (m, 2H), 7.06 (m, 2H), 6.70 (m, 1H), 6.45-6.00 (m, 5H), 5.66-5.36 (m, 5H ): LCMS (m/z) ES− 1168 (M−1)⁺.

### Example 11: Synthesis of Compound A14

To a solution of 40-O-(prop-2-ynyloxy) rapamycin A1 (200 mg, 0.2 mmol) and 1-(azido-methyl)-4-fluorobenzene (100 mg, 0.6 mmol) in anhydrous THF (9 mL) was added DIPEA (100 μL, 0.6 mmol) and CuI (20 mg, 0.1 mmol) under N₂. The solution was stirred at rt for 3 hours. Then, 20 mL water was added and extracted with EtOAc (20 mL×3). The combined organic layer was washed by brine and dried over anhydrous Na₂SO₄. After concentration, the residue was purified with silica gel chromatography (30% to 100% of EtOAc in petroleum ether as eluent) to give white solid which was further purified by prep-HPLC to give compound A14 (32 mg, 14%) as a white solid. ¹H NMR (300 MHz, CDCl₃) δ7.58 (m, 1H), 7.26 (m, 2H), 7.06 (m, 2H), 6.75 (m, 1H), 6.50-6.00 (m, 5H), 5.60-5.36 (m, 5H): LCMS (m/z) ES=1102 (M−1).

### Example 12: Synthesis of Compound A15

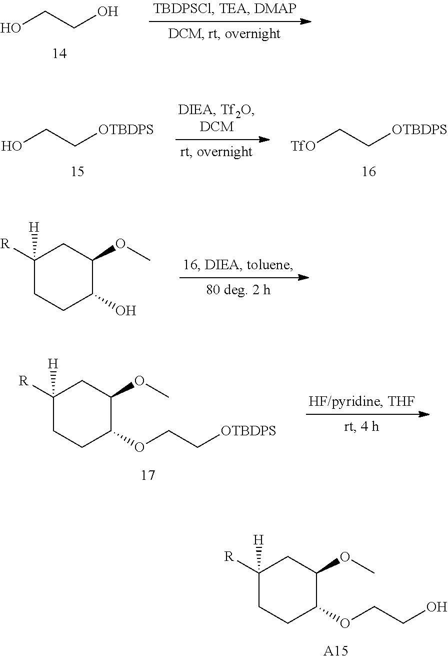

### Preparation of Intermediate 15

To a solution of compound 14 (3 g, 48 mmol) and Et₃N (5 g, 50 mmol) in DCM (100 mL) was added DMAP (0.6 g, 5 mmol) and dropwised TBDPS-CI (4.4 g, 16 mmol) at 0 deg and stirred at rt overnight. Then, 100 mL water was added and extracted with DCM (80 mL×3). The combined organic layer was washed by brine and dried over anhydrous Na₂SO₄. After concentration, the residue was purified with silica gel chromatography (30% of EtOAc in petroleum ether as eluent) to give intermediate 15 (1.7 g, 12%) as a colorless oil. ¹H NMR (300 MHz, CDCl₃) δ7.63 (m, 4H), 7.36 (m, 6H), 3.74 (m, 2H), 3.66 (m, 2H), 1.04 (s, 9H): LCMS (m/z) ES+ 301 (M+1)⁺.

### Preparation of Intermediate 16

To a solution of intermediate 15 (1.7 g, 5.7 mmol) and DIPEA (1.5 g, 11.4 mmol) in DCM (40 mL) was added Tf₂O (1.7 g, 6 mmol) at 0 deg and stirred at rt overnight. Then, 50 mL water was added and extracted with DCM (40 mL×3). The combined organic layer was washed by brine and dried over anhydrous Na₂SO₄. After concentration, the residue was purified with silica gel chromatography (10% of EtOAc in petroleum ether as eluent) to give intermediate 16 (1.5 g, 63%) as a colorless oil. ¹H NMR (300 MHz, CDCl₃) δ7.63 (m, 4H), 7.36 (m, 6H), 4.58 (m, 2H), 3.95 (m, 2H), 1.04 (s, 9H): LCMS (m/z) ES+ 433 (M+1)⁺.

### Preparation of Intermediate 17

To a solution of Rapamycin (400 mg, 0.43 mmol) and DIPEA (278 mg, 2.15 mmol) in toluene (30 mL) was added intermediate 16 (0.93 g, 2.15 mmol) at rt and stirred at 80 deg for 2 hours. Then, 50 mL water was added and extracted with EtOAc (30 mL×3). The combined organic layer was washed by 0.5 N HCL, saturated NaHCO₃ and brine, dried over anhydrous Na₂SO₄. After concentration, the residue was purified with silica gel chromatography (25% to 40% of EtOAc in petroleum ether as eluent) to give intermediate 17 (280 mg, 53%) give as a white solid. LCMS (m/z) ES− 1194 (M−1)⁻.

### Preparation of Compound A15

To a solution of intermediate 17 (280 mg, 0.23 mmol) in THF (10 mL) was added 2 mL HF in pyridine at 0 deg and stirred at rt for 4 hours. Then, 20 mL water was added and extracted with EtOAc (20 mL×3). The combined organic layer was washed by 0.5N HCl, saturated NaHCO₃ and brine, dried over anhydrous Na₂SO₄. After concentration, the residue was purified with silica gel chromatography (30% to 100% of EtOAc in petroleum ether as eluent) to give white solid which was further purified by prep-HPLC to give compound A15 (34 mg, 15%) as a white solid. ¹H NMR (300 MHz, CDCl₃) δ6.40-6.00 (m, 5H), 5.53-5.25 (m, 4H), 4.83 (s, 1H), 4.13 (m, 1H): LCMS (m/z) ES− 957 (M−1)⁻.

### Synthesis of Series B of Rapamycin Derivatives of the Present Invention

B series of rapamcin derivatives were prepared according to the following reaction scheme:

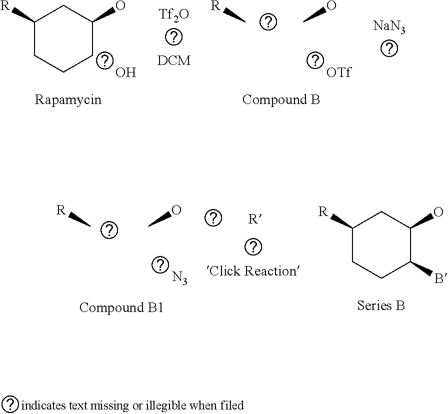

In the formula of the schemes shown above, R and B′ have the following structures in some examples of the B series of rapamycin derivatives.

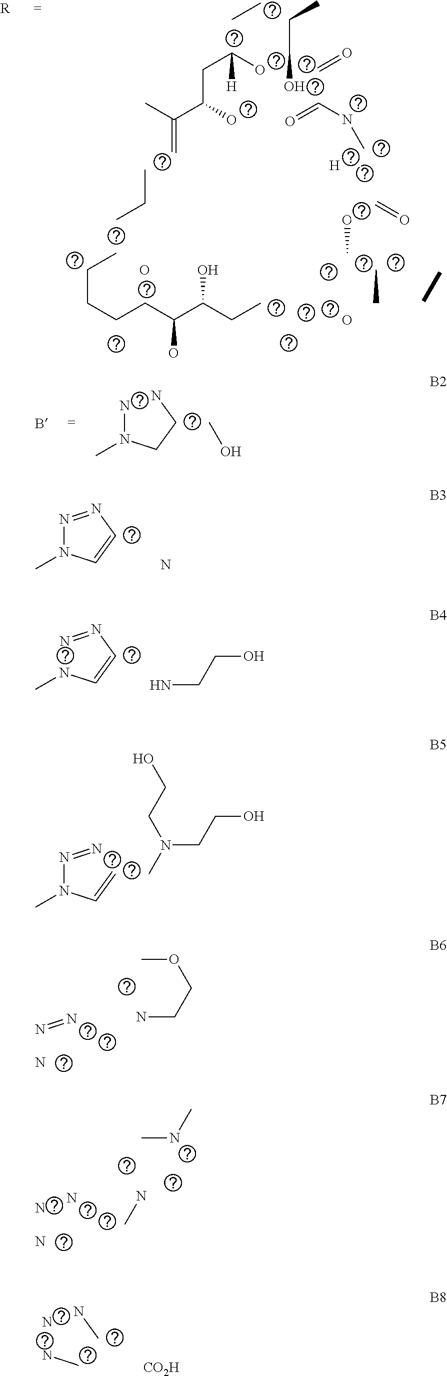

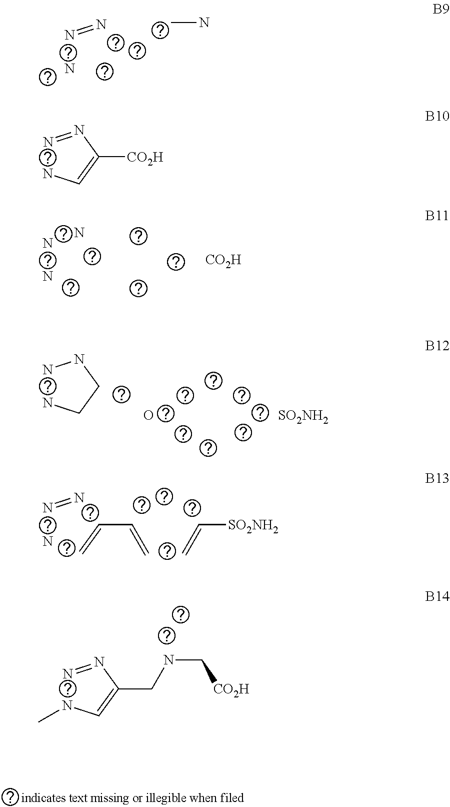

### Example 13: Synthesis of Compound B1

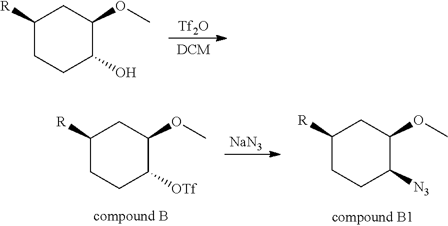

To a solution ofrapamycin (5 g, 5.5 mmol) and 2,6-di-tert-butyl-4-methylpyridine (3.4 g, 3.4 mmol) in dried DCM (150 mL), tritluoromethanesulfonic anhydride (1.55 g, 5.5 mmol) was added at 0 oC. After the mixture was stirred for 2 h at room temperature, NaN₃ (3.6 g, 55 mmol) was added and DMSO (60 mL) was added at −10 oC, the mixture was stirred for at 40 oC for 5 h. The mixture was quenched by addition of water, extracted with DCM (200 mL×2), and the combined extracts were washed with water, dried over Na₂SO₄, and evaporated to dryness in vacuo. The crude product was purified by column chromatography (25% of EtOAc in petroleum ether as eluent) to give compound B (1.5 g, 30%) as a white solid. LCMS (m/z) ES− 937 (M−H)⁻.

### Example 14: Synthesis of Compound B2

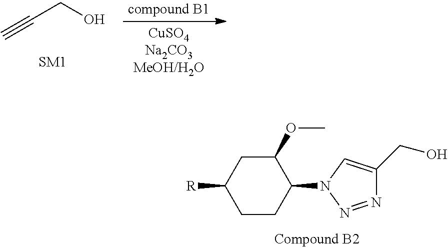

To a solution of compound B1 (150 mg, 0.16 mmol) and SM1 (27 mg, 0.48 mmol) in McOH/H₂O (4 mL/2 mL) was added vitamine C sodium salt (63 mg, 0.32 mmol), followed with the addition of CuSO₄ (51 mg, 0.32 mmol) and Na₂CO₃(51 mg, 0.48 mmol). After stirred overnight, the mixture was filtered, the filtrate was concentrated and purified by column chromatography (0 to 2% of methanol in dichloromethane as eluent) to give Compound B2 (33.2 mg, 21%) as yellow solid. ¹H NMR (300 MHz, CDCl₃) δ7.83 (m, 1H), 6.37-6.01 (m, 4H), 5.40-5.31 (m, 4H): LCMS (m/z) ES− 993 (M−H)⁻.

### Example 15: Synthesis of Compound B3

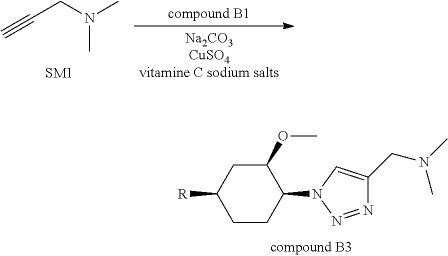

To a solution of compound B1 (150 mg, 0.16 mmol) and SM1 (40 mg, 0.48 mmol) in MeOH/H₂O (4 mL/2 mL) was added vitamine C sodium salt (63 mg, 0.32 mmol) followed with the addition of CuSO₄ (51 mg, 0.32 mmol) and Na₂CO₃ (51 mg, 0.48 mmol). After stirred overnight, the mixture was filtered, the nitrate was concentrated and was purified by column chromatography (0 to 2% of methanol in dichloromethane as eluent) to give Compound B3 (20.7 mg, 13%) as white solid. ¹H NMR (300 MHz, CDCl₃) δ8.55 (s, 1H), 7.89 (m, 1H), 6.39-6.02 (m, 4H), 5.46-4.83 (m, 4H): LCMS (m/z) ES− 1020 (M−H)⁻.

### Example 16: Synthesis of Compound B4

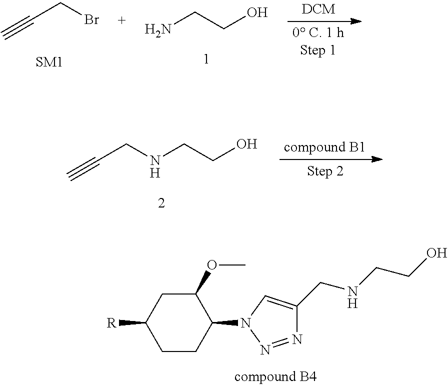

To a solution of 1 (2 g, 32.8 mmol) in DCM (100 mL) was added SM1 (2 g, 16.4 mmol) at 0 oC dropwise over 1 hour. The mixture was concentrated and the residue was purified by column chromatography (0 to 2% of methanol in dichloromethane as eluent) to give intermediate 2 (0.9 g, 54%) as yellow oil. LCMS (m/z) ES+ 100 (M+H)⁺.

To a solution of compound B1 (150 mg, 0.16 mmol) and intermediate 2 (48 mg, 0.48 mmol) in MeOH/H₂O (4 mL/2 mL) was added vitamine C sodium salt (63 mg, 0.32 mmol) followed with the addition of CuSO₄ (51 mg, 0.32 mmol) and Na₂CO₃ (51 mg, 0.48 mmol). After stirred overnight, the mixture was filtered, the filtrate was concentrated and purified by column chromatography (0 to 2% of methanol in dichloromethane as eluent) to give Compound B4 (17.1 mg, 1 1%) as white solid. ¹H NMR (300 MHz, CDCl₃) δ8.40 (s, 1H), 6.39-6.02 (m, 4H), 5.37-4.94 (m, 4H): LCMS (m/z) ES− 1036 (M−H)⁻.

### Example 17: Synthesis of Compound B5

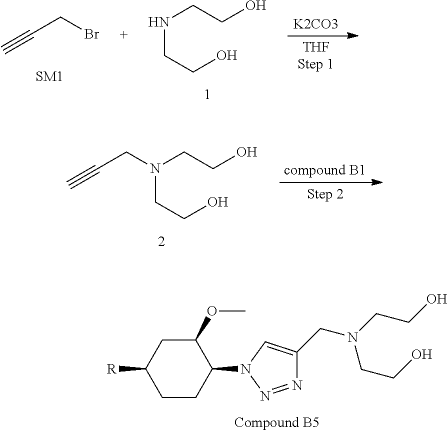

To a solution of 1 (2 g, 19.0 mmol) in THF (40 mL) was added K₂CO₃ (5.2 g, 38 mmol) and SM1 (2.2 g, 19 mmol) at 0° C. After stirred for 4 h at r.t. the mixture was filtered, the filtrate was concentrated and purified by column chromatography (0 to 3% of methanol in dichloromethane as eluent) to give intermediate 2 (1.2 g, 44%) as yellow oil. ¹H NMR (300 MHz, CDCl₃) δ3.65 (m, 4H), 3.49 (m, 2H), 2.75 (m, 4H), 2.22 (m, 1H).

To a solution of compound B1 (150 mg, 0.16 mmol) and intermediate 2 (69 mg, 0.48 mmol) in MeOH/H₂O (4 mL/2 mL) was added vitamine C sodium salt (63 mg, 0.32 mmol) followed with the addition of CuSO₄ (51 mg, 0.32 mmol) and Na₂CO₃ (51 mg, 0.48 mmol). After stirred overnight, the mixture was filtered, the filtrate was concentrated and purified by column chromatography (0 to 2%) of methanol in dichloromethane as eluent) to give Compound B5 (26.7 mg, 11%) as white solid. ¹H NMR (300 MHz, CDCl₃) δ7.92 (s, 1H), 6.39-5.99 (m, 4H), 5.45-4.84 (m, 4H): LCMS (m/z) ES− 1080 (M−H)⁻.

### Example 18: Synthesis of Compound B6

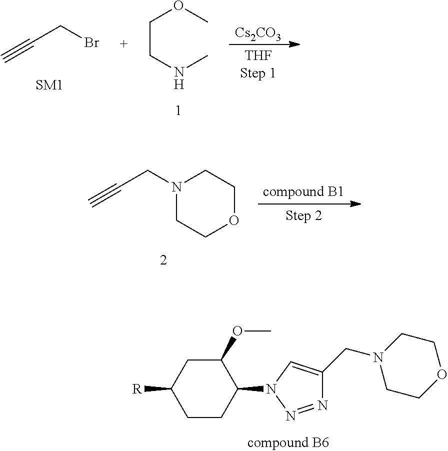

To a solution of 1 (0.5 g, 5.8 mmol) in THIF (40 mL) was added K₂CO₃ (1.6 g, 11.6 mmol) and SM1 (0.69 g, 5.8 mmol) at 0 oC. After stirred for 4 h at r.t. the mixture was filtered, the filtrate was concentrated and purified by column chromatography (3%) of methanol in dichloromethane as eluent) to give 2 (0.3 g, 42%) as yellow oil. LCMS (m/z) ES+ 125 (M+H)⁺.

To a solution of compound B1 (150 mg, 0.16 mmol) and 2 (60 mg, 0.48 mmol) in MeOH/H₂O (4 mL/2 mL) was added vitamine C sodium salt (6.3 mg, 0.32 mmol) followed with the addition of CuSO₄ (51 mg, 0.32 mmol) and Na₂CO₃ (51 mg. 0.48 mmol). After stirred overnight, the mixture was filtered, the filtrate was concentrated and purified by column chromatography (0 to 2%) of methanol in dichloromethane as eluent) to give Compound 136 (25.8 mg, 15%) as white solid. ¹H NMR (300 MHz, CDCl₃) δ7.81 (s, 1H), 6.40-6.02 (m, 411), 5.45-4.81 (m, 1H): LCMS (m/z) ES− 1062 (M−H)⁻.

### Example 19: Synthesis of Compound B7

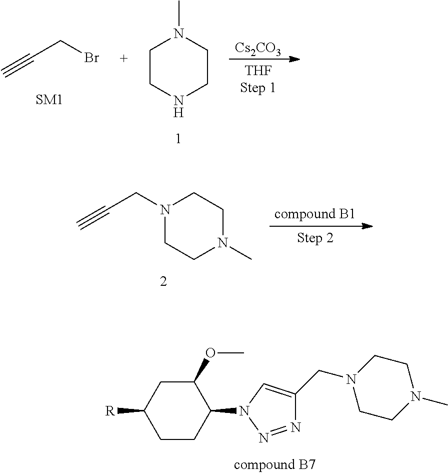

To a solution of 1 (0.5 g, 5 mmol) in THF (40 mL) was added K₂CO₃ (1.4 g, 10 mmol) and SM1 (0.6 g, 5 mmol) at 0° C. After stirred for 4 h at r.t. the mixture was filtered, the filtrate was concentrated and purified by column chromatography (3% of methanol in dichloromethane as eluent) to give intermediate 2 (0.5 g, 72%) as yellow oil. LCMS (m/z) ES+ 139 (M+H)⁺.

To a solution of compound B1 (150 mg, 0.16 mmol) and 2 (60 mg, 0.48 mmol) in MeOH/H₂(4 mL/2 mL) was added vitamine C sodium salt (63 mg, 0.32 mmol) followed with the addition of CuSO₄ (51 mg, 0.32 mmol) and Na₂CO₃ (51 mg, 0.48 mmol). After stirred overnight, the mixture was filtered, the filtrate was concentrated and purified by column chromatography (2% of methanol in dichloromethane as eluent) to give Compound B7 (12 mg, 7%) as white solid. ¹H NMR (300 MHz, CDCl₃) δ8.39 (s, 1H), 7.76 (s, 1H), 6.37-6.01 (m, 4H), 5.41-4.78 (m, 4H): LCMS (m/z), ES− 1075 (M−H)⁻.

### Example 20: Synthesis of Compound B9

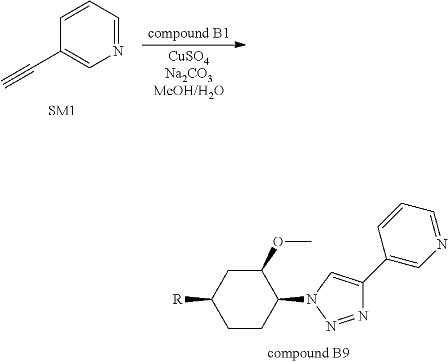

To a solution of compound B1 (150 mg, 0.16 mmol) and SM1 (50 mg, 0.48 mmol) in MeOH/H₂O (4 mL/2 mL) was added vitamine C sodium salt (63 mg, 0.32 mmol) followed with the addition of CuSO₄ (51 mg, 0.32 mmol) and Na₂CO₃ (51 mg, 0.48 mmol). Alter stirred overnight, the mixture was filtered, the filtrate was concentrated and purified by column chromatography (2% of methanol in dichloromethane as eluent) to give Compound B9 (37.1 mg, 22%) as white solid. ¹H NMR (300 MHz, CDCl₃) δ9.37-7.89 (m, 5H), 6.39-6.01 (m, 4H), 5.42-4.99 (m, 4H): LCMS (m/z) ES− 1040 (M−H)⁻.

### Example 21: Synthesis of Compound B11

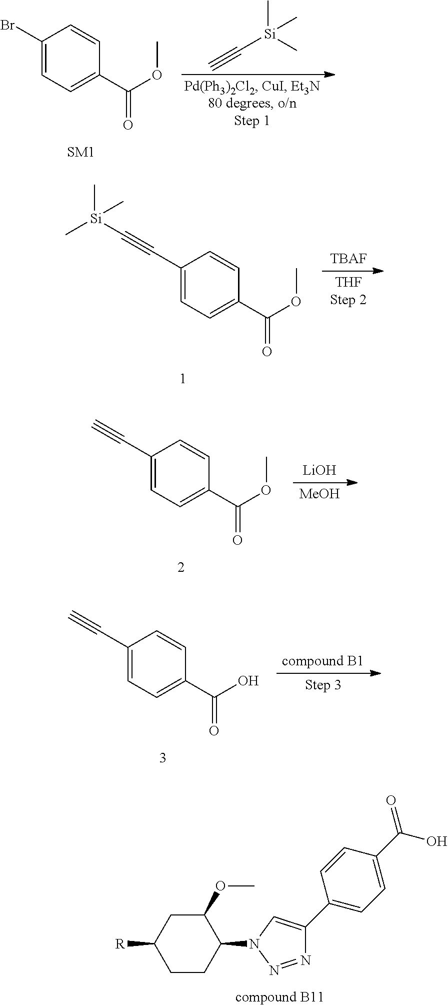

To a solution of SM1 (2.1 g, 10 mmol) in Dioxane (20 mL) was added ethynltrimethylsilane (2 g, 20 mmol), CuI (191 mg, 1 mmol) and Pd(PPH₃)₂Cl₂ (730 mg, 1 mmol) under N₂, then Et₃N (10 g, 100 mmol) was added dropwise. After stirred at 100 oC overnight, the mixture was quenched by water, extracted with EtOAc (50 mL×2). The combine organic layer was dried over anhydrous Na₂SO₄, concentrated to give crude intermediate 1.

The crude intermediate 1 was dissolved treated with TBAF in THF (20 mL, 20 mmol) at r.t. for 2 hours, then quenched by water and extracted with EtOAc (50 mL×2). The combine organic laser was dried over anhxdrous Na₂SO₄, concentrated to give crude intermediate 2 which was purified by column chromatography to give 2 (0.9 g, 58%)) as yellow solid. ¹H NMR (300 MHz, DMSO-d6) δ7.96 (d, 2H), 7.62 (d, 2H), 4.48 (s, 1H), 3.87 (s, 3H),

To a solution of 2 (0.5 g, 3.13 mmol) in MeOH (10 mL) was added LiOH (0.312 g, 12.52 mmol) in water (10 mL). The mixture was stirred for 2 h, then quenched by HCl solution (2N), extracted with EtOAc (30 mL*3). The combined organic was dried over anhydrous Na₂SO₄, concentrated to gie the desired intermediate 3 (0.35 g, 77%) as yellow solid.

To a solution of compound B1 (150 mg, 0.16 mmol) and intermediate 3 (70 mg, 0.48 mmol) in MeOH/H₂O (6 mL/3 mL) was added vitamine C sodium salt (63 mg, 0.32 mmol) followed with the addition of CuSO₄ (51 mg, 0.32 mmol) and Na₂CO₃ (51 mg, 0.48 mmol). After stirred overnight, the mixture was adjusted to pH about 3-4, filtered, the filtrate was concentrated and purified by column chromatography (1.5% of methanol in dichloromethane as eluent) to give Compound B11 (15.5 mg, 9%) as white solid. ¹H NMR (300 MHz, CDCl₃) δ8.19 (m, 3H), 7.98 (d, 2H), 6.77-6.11 (m, 4H), 5.49-4.51 (m, 4H): LCMS (m/z) ES− 1083 (M−H)⁻.

### Example 22: Synthesis of Compound B12

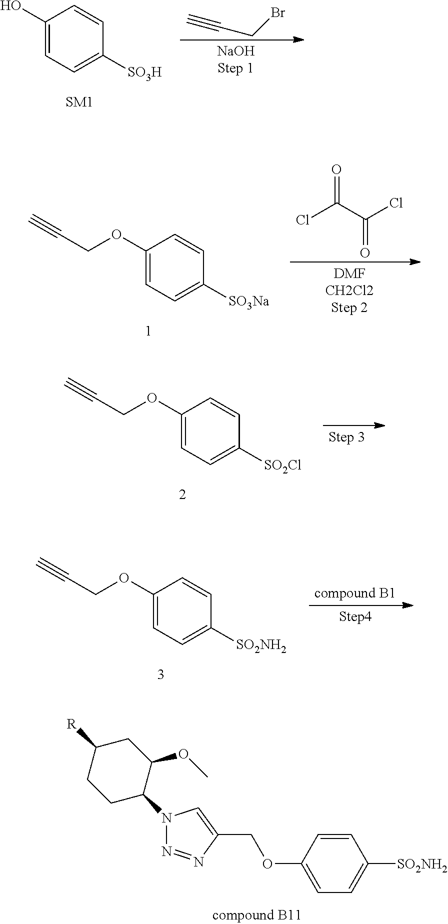

To a solution of SM1 (4 g, 23 mmol) in propan-2-ol (20 mL) was added NaOH (2.8 g, 69 mmol) in water and 3-bromoprop-1-yne (2.4 g, 20 mmol). After stirred lor 4 h at 70° C., the mixture was concentrated, filtered, the filter cake was washed by water, dried to give intermediate 1 (2 g, 37%) as yellow solid. LCMS (m/z) ES° 234 (M+Na)⁺.

To a solution of intermediate 1 (1 g, 4.3 mmol) in DMF (8 mL) was added oxalyl dichloride (1.1 g, 8.6 mmol) in DCM (4 mL) dropwise at 0° C. After stirred overnight at r.t. the mixture was quenched by water, extracted with DCM (30 mL×3). The combined organic was dried over anhydrous Na₂SO₄, concentrated, purified by column chromatography (0 to 10% of EtOAc in petroleum ether as eluent) to give the desired intermediate 2 (0.35 g, 77%) as yellow solid.

Intermediate 2 (0.35 g, 1.5 mmol) was added into ammonia water (5 mL), the mixture was stirred for 1 h at r.t., quenched by addition of water, extracted with EtOAc (20 mL×2). The combined organic was dried over anhydrous Na₂SO₄, concentrated, purified by column chromatography (0 to 50% of EtOAc in petroleum ether as eluent) to give intermediate 3 (0.2 g, 20% for two steps) as yellow solid. ¹H NMR (300 MHz, DMSO-d6) δ7.77 (d, 2H), 7.24 (s, 2H), 7.14 (d, 2H), 4.90 (d, 2H), 3.63 (m, 1H).

To a solution of compound B1 (150 mg, 0.16 mmol) and intermediate 3 (101 mg, 0.48 mmol) in MeOH/H₂O (6 mL, 3 mL) was added vitamine C sodium salt (63 mg, 0.32 mmol) followed with the addition of CuSO₄ (51 mg, 0.32 mmol) and Na₂CO₃ (51 mg, 0.48 mmol). After stirred overnight, the mixture was filtered, and the filtrate was concentrated and purified by column chromatography (0 to 2.5% of methanol in dichloromethane as eluent) to give Compound B 12 (13.4 mg, 7.3%) as white solid. ¹H NMR (300 MHz, CDCl₃) δ7.88 (m, 3H), 7.12 (m, 2H), 6.39-6.01 (m, 4H), 5.42-4.63 (m, 4H): LCMS (m/z) ES− 1148 (M−H)⁻.

### Example 23: Synthesis of Compound B13

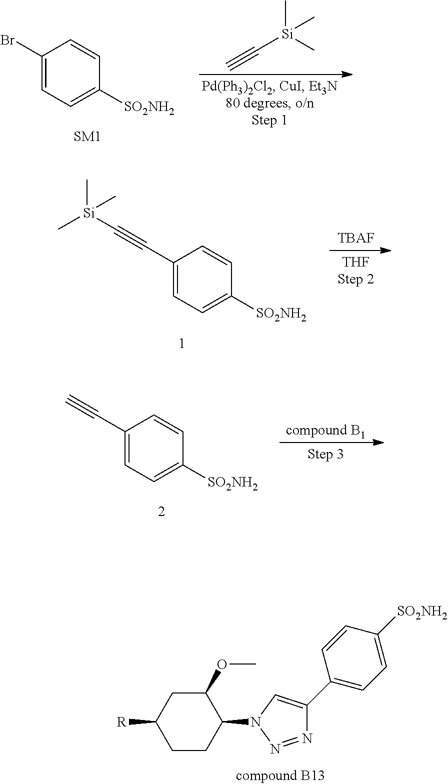

To a solution of SM1 (2.3 g, 10 mmol) in dioxane (20 mL) was added ethynyltrimethylsilane (2 g, 20 mmol), CuI (191 mg, 1 mmol) and Pd(PPh₃)₂Cl₂ (730 mg, 1 mmol) under N₂, then Et₃N (10 g, 100 mmol) was added dropwise. After stirred overnight at 100° C. the mixture was quenched by addition of water, the mixture was extracted with EtOAc (50 mL×2). The combine organic layer was dried over anhydrous N₂aSO₄, concentrated to give crude intermediate 1.

The crude intermediate 1 was treated with TBAF in THF (20 mL, 20 mmol) and stirred for 2 h at r.t., then quenched by water and extracted with EtOAc (50 mL×2). The combine organic layer was dried over anhydrous Na₂SO₄, concentrated to give crude intermediate 2, which was purified by column chromatography to give pure intermediate 2 (1.5 g, 84%) as yellow solid. ¹H NMR (300 MHz, DMSO-d6) δ7.82 (d, 2H), 7.68 (d, 2H), 7.46 (s, 2H), 4.45 (s,

To a solution of compound B1 (150 mg, 0.16 mmol) and intermediate 2 (87 mg, 0.48 mmol) in MeOH/H₂O (6 mL/3 mL) was added vitamine C sodium salt (63 mg, 0.32 mmol) followed with the addition of CuSO₄ (51 mg, 0.32 mmol), Na₂CO₃ (51 mg, 0.48 mmol). After stirred at r.t. overnight, the mixture was filtered. The filtrate was concentrated and purified by column chromatography (0 to 1.5% of methanol in dichloromethane as eluent) to give Compound B13 (48.7 mg, 27%) as white solid. ¹H NMR (300 MHz, CDCl₃) δ8.21 (m, 1H), 7.93 (m, 4H), 6.37-6.00 (m, 4H), 5.44-5.30 (m, 5H), LCMS (m/z) ES− 1118 (M−H)⁻.

### Example 24: Synthesis of Compound B14

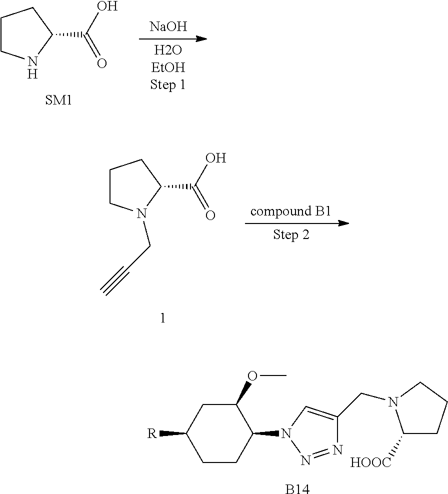

To a solution of SM1 (2.3 g, 20 mmol) in EtOH (20 mL) was added NaOH (1.6 g, 40 mmol) in water, then 3-bromoprop-1-yne (2.4 g, 20 mmol) was added at 0° C. After stirred for 4 h at r.t. the mixture was quenched by HCl solution (2N) and pH was adjusted to 3˜4, extracted with EtOAc (50 mL*5). The combined organic was dried over anhydrous Na₂SO₄, concentrated, purified by column chromatography (0-3% of methanol in dichloromethane as eluent) to give intermediate 1 (1.2 g, 40%) as colorless crystal. LCMS (m/z) ES− 154 (M−H)⁻.

To a solution of compound B1 (150 mg, 0.16 mmol) and intermediate 1 (7.3 mg, 0.48 mmol) in MeOH/H₂O (4 mL/2 mL) was added vitamine C sodium salt (63 mg, 0.32 mmol) followed with the addition of CuSO₄ (51 mg, 0.32 mmol) and Na₂CO₃ (51 mg, 0.48 mmol). After stirred overnight, the mixture was filtered, the filtrate was concentrated and purified by column chromatography (2% of methanol in dichloromethane as eluent) to give Compound B14 (12.6 mg, 7.2%) as white solid. ¹H NMR (300 MHz, CDCl₃) δ8.17 (m, 1H), 6.39-5.99 (m, 4H), 5.56-4.87 (m, 4H): LCMS (m/z) ES− 1090 (M−H)⁻.

Enzymatic activities of rapamycin analogs of the present invention

The mTOR is a serine/threonine protein kinase that has been shown to regulate multiple cellular responses including cell growth, proliferation, motility, survival and protein synthesis, mTOR kinase activity is regulated by several upstream signaling pathways and its dysregulation has been implicated in several forms of cancer. Now we use a Terbium labeled anti-phosphorylated 4E--BP1 antibody to detect phosphorylation of the GFP-labeled substrate by mTOR. This TR-FRET based assay can be used to screen inhibitors of mTOR in vitro.

Materials: Assay buffer components: 1M HEPES pH7.5, GIBCO, Cat#15630: 1M MgCl₂, Sigma, Cat# M1028: 0.5M EDTA, GIBCO, Cat# 15575: DTT, Sigma Cat# 43819: EGTA, Sigma Cat# E3889; Triton X100, Sigma, Cat# T8787: USA CALBIOCHEM Cat# 126575.

Enzyme, substrate and detection reagents: mTOR: Invitrogen, Cat# PV4753: GFP-4E-BPI: Invitrogen, Cat# PV4759: FKBP12: SinoBiological, Cat# 10268-H08E: ATP: Sigma Cat# A26209: Tb-anti-p4E-BP1: Invitrogen, Cat# PV4755: TR-FRET Dilution buffer Invitrogen, Cat# PV3574.

Plate: Compounds preparation plate: 384-well, Corning cat# 3657: Assay plate: black low volume 384 well microtiter plate (Greiner Bio-One, Cat# 784076).

Procedure: Compounds dosage gradient solution preparation:

Compounds were 3-fold serial diluted in 100% DMSO in a microtiter plate (Corning 3674) at 11 different concentrations in the range of 100 μM to 1.7 nM (100 μM, 33 μM, 11 μM, 3.7 μM, 1.2 μM, 411 nM, 137 nM, 46 nM, 15 nM, 5 nM, 1.7 nM). Then the diluted compounds in 100% DMSO was 10-fold diluted with ddH2O, so the compounds were in 10% DMSO.

A typical assay protocol of measuring the mTOR inhibitory ability of the rapamycin derivatives of the invention is as follows:

### Assay Protocol:

0.5 μl diluted compounds in 10% DMSO was pipetted into a black low volume 384 well microtiter plate (Greiner Bio-One, Frickenhausen, Germany, cat# 784076):
2 μl of a solution of mTOR in aqueous assay buffer |50 mM HEPES/NaOH pH 7.5. mM MgCl12, 1.0 mM dithiothreitol, 1 mM EGTA, 0.01% (v/v) Triton-X100 (Sigma), 0.01% (w/v| bovine serum albumine (BSA)| (mTOR, 0.3125 ng/μl=>final cone, in the 5 μl assay volume is 0.125 ng/μl) were added to the assay plate and the compound-enzyme mixture was incubated for 15 min at 22° C. to allow pre-binding of the test compounds to the enzyme before the start of the kinase reaction:

The kinase reaction was started by the addition of 2.5 μl of a solution of ATP (ATP, 200 μM=>final conc., in the 5 μl assay volume is 10 μM) and substrate (0.8 μM=>final conc. in the 5 μl assay volume is 0.4 μM) in assay buffer and the resulting mixture was incubated for 18 min at 22° C.

The reaction was stopped by the addition of 5 ul of 30 mM EDTA (EDTA, 30 mM=> final cone, in the 10 ∥l assay volume is 15 mM) and 4 nM Tb-chelate labeled anti-4E-BP1 |pT46| phosphospecific antibody |Invitrogen Cat# PV4755| (Tb-labeled antibody, 4 nM=>final conc. in the 10 μl assay volume is 2 nM) in TR-FRET dilution buffer, the resulting mixture was incubated 1 hour at 22° C. to allow the formation of complex of the phosphorylated substrate and the Tb-chelate labeled antibody.

The amount of phosphorylated substrate was evaluated by measurement of the resonance energy transfer from the Tb-chelate to the GFP. Therefore, the fluorescence emission at 495 nmn and 520 nm after excitation at 340 nm was measured on envision 2104 multilabel reader (Perkin-Elmer). The ratio of the emission at 520 nm and at 495 nm was taken as the measure for the amount of phosphorylated substrate. The data were normalised (enzyme reaction without inhibitor=0% inhibition, all other assay components but no enzyme=100% inhibition) and IC50 values were calculated by a 4 parameter fit (equation (1)) using IDBS XLfit software (ID Business Solutions Ltd., UK)

Y=Bottom+(TOP−Bottom)/(1+10̂((LogIC50−X)*hillslope)) equation (1)

In this equation, Y was the normalized % inhibition value. X was the log value of the test compound concentration. IC50 was the concentration of compound where half of maximal inhibition was achieved.

The testing results of mTOR inhibitory effects of the rapamycin analogs/derivatives of the invention are shown below:

| Compound ID | IC50(nM) | Compound ID | IC50(nM) |
|---|---|---|---|
| Rapamycin | 5.09 | | |
| Compound A15 | 6.43 | B2 | 18.43 |
| Compound A4 | 6604.427021 | B3 | 15.40 |
| Compound A7 | >10000 | B4 | 28.52 |
| Compound A10 | >10000 | B5 | 19.38 |
| Compound A5 | >10000 | B6 | 12.96 |
| Compound A6 | 9143.20 | B7 | 23.77 |
| Compound A3 | >10000 | B9 | 27.16 |
| Compound A13 | >10000 | B11 | 446.00 |
| Compound A12 | >10000 | B12 | 35.19 |
| Compound A14 | >10000 | B13 | 29.03 |
| Compound A9 | >10000 | B14 | 6.80 |

### Tumor Cell Inhibition Studies:

### Cell Proliferation Assay

The effect of different compounds on the cellular activities was quantitated through determining the number of living cells in a culture by a homogeneous detection method for quantitative determination of cell viability by the CellTiter-Glo® chemiluminescence detection kit for ATP. ATP is an indicator of the metabolism of living cells. Homogeneous detection step is added directly to the single reagent (CellTiter-Glo® Reagent) in serum-containing cultured cells, without washing the cells or removing the medium. After adding reagent and mixing in a 96-well or 384-well plates, the number of cells that can be quantified by the system within 10 minutes, is as low as 15 cells in each well.

### Preparation of the Reagents

Different cell types were cultured using a medium, containing 10% FBS plus 1% penicillin streptomycin double antibiotics, and the following appropriate additives: DMEM medium (Gibco, Item No. 11995073) for culturing colorectal cancer cells HCT116, breast cancer cells MCF-7 and MDA-MB-231 melanoma cells SK-MEL-28, A549 and epidermal squamous cell carcinoma cell A431: RPMI-1640 medium (containing 2 mM L-glutamine, 1.5 g/L sodium bicarbonate, 4.5 g/L glucose, 10 mM HEPES, 1.0 mM sodium pyruvate (Item # 72400-120 from Gibco) for culturing U87/MG and kidney cancer786-O: F-12K mixed medium (Item #2127 from Gibco) for culturing prostate cancer cell line PC-3.

### Instrumentation

Multi-Label Micro-Plate Reader Envision 214 from Perkin Elmer

### Cell Culture Conditions

All 9 cell lines were cultured in the wells in the plates, at a cell density of 3000 cells/well after 9 passages.

### Preparation of Culture Media and Cell Culture Conditions:

Prepare the compounds and condition the cells the next day: each chemical compound to be assayed was diluted to 10 mM stock solution with 100% DMSO, followed by additional dilution with 100% DMSO diluted to 2 mM, followed by serial 5× dilution using serum-free cell culture medium to a final 10 different diluted concentrations points (2000, 400, 80, 16, 3.2, 0.64, 0.128, 0.0256, 0.00512, 0.00102 μM), plus 0.5% DMSO (no compound) as a maximum control and 10 μM Rapamycin as a minimum control. A solution of 0.5 μl of each diluted compound is added to the 100 μl of cell culture plate, the final compound concentration of 10 points (10.2, 0.4, 0.08, 0.016, 0.0032, 0.00064, 0.000128, 0.0000256, 0.00000512 μM). The cells were then cultured in 37° C. incubator for 72 hours. In order to ensure the reliability of the experiments of determining the inhibition of each compound, a duplicate was used for each compound concentration gradient will do two repeated (Table 1), and the determination of each compound was repeated twice.

### Plate Reading

After 72-hr of cell culture, 50 ul of CellTiter Glo was added to each well on the plate, and shaken for 5 min on a shaker followed by 10 min at room temperature. The cell number was analyzed by the Micro-Plate reader.

### Data Analysis:

Cell viability was obtained through the reading by the multi-label micro-plate reader. The effect of each dilution value on the % cell viability was calculated using the following formula: % cell inhibition−100-100×(Signal-low control)/(High control-low control, in which signal, low control, and high control are the test compound, minimal value, and maximal value respectively.

The IC50 value of each test compound in inhibiting the cells is obtained by formula 2 (below):

Y Bottom+(TOP−Bottom)/(1+((IC50/X)\textcircled{}hillslope)), in which X and y are known values, IC50, Hillslope, Top and Bottom 4 parameters generated by the analysis software, Y as the % inhibition, X as the test compound concentration, and IC50 as the concentration of the compound needed to inhbit 50% of the cells. Hillslope is the slope of curve fitting, usually around 1.

All experimental data were analyzed by IDBS X1.fit5 (ID Business Solutions Ltd., UK).

### EXPERIMENTAL RESULTS AND CONCLUSION

All the potency of each lest compound is shown in one of the following graphs for each of the cancer cell models tested. The lower the curve in the graph, the more potent each compound is. From the data shown in each of the graphs, it is clear that all the series B compounds showed varying levels of high potency against the cancer cell tested. Some of the B series compounds were extremely potent, reaching a potency level of nM concentration range. Renal cell carcinoma tumor cell inhibition studies:

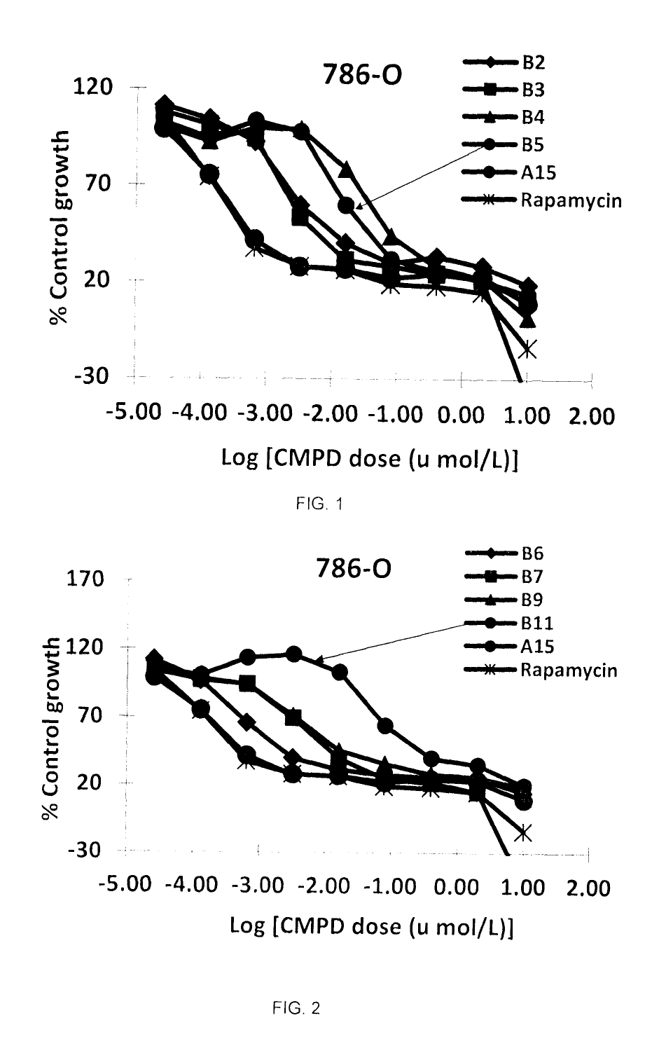

Renal cell carcinoma tumor cell inhibition studies: FIG. 1 and FIG. 2.

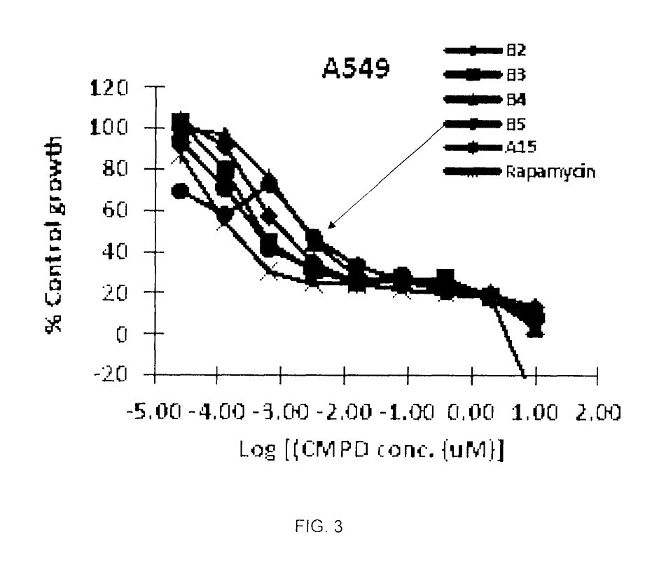

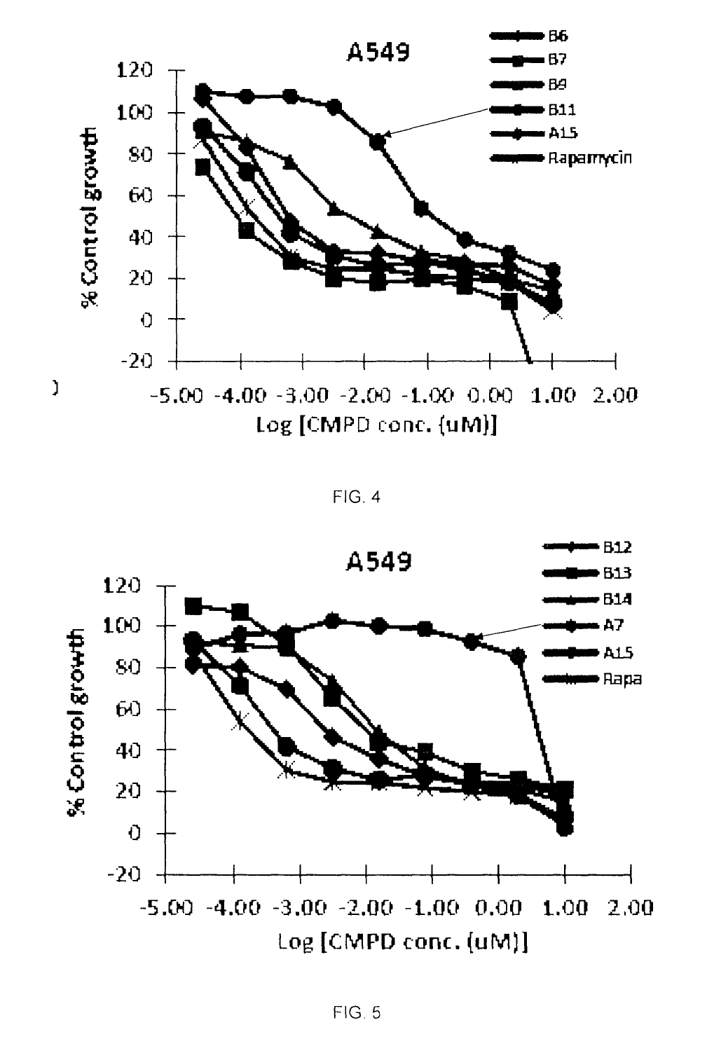

Lung Cancer A549 cell inhibition studies: FIG. 3, FIG. 4 and FIG. 5.

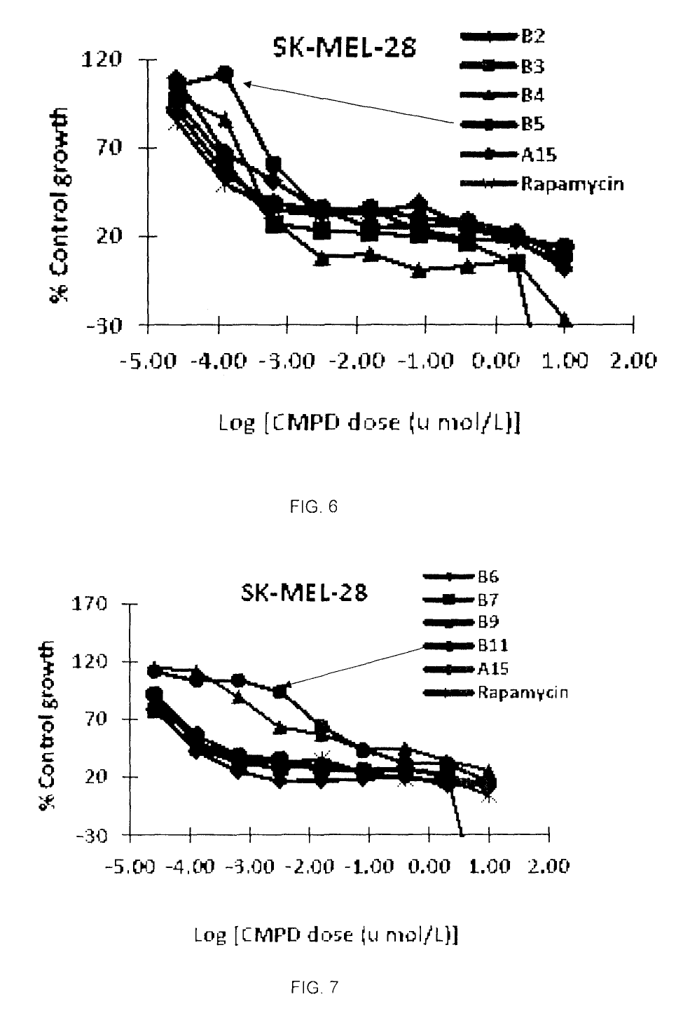

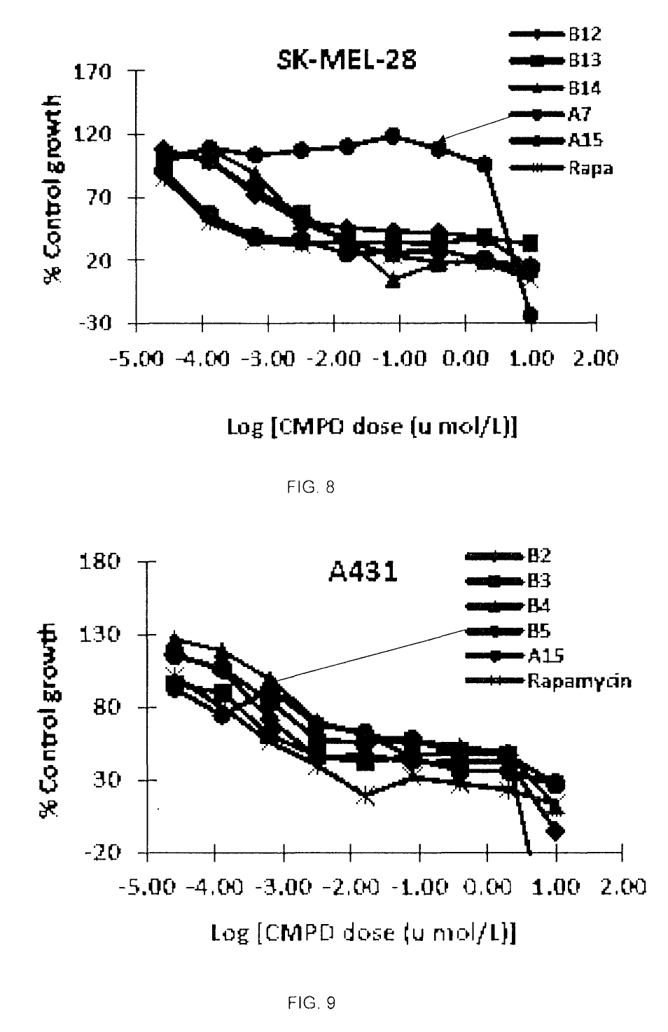

Melanoma SK-MEL-28 cell inhibition studies: FIG. 6, FIG. 7 and FIG. 8.

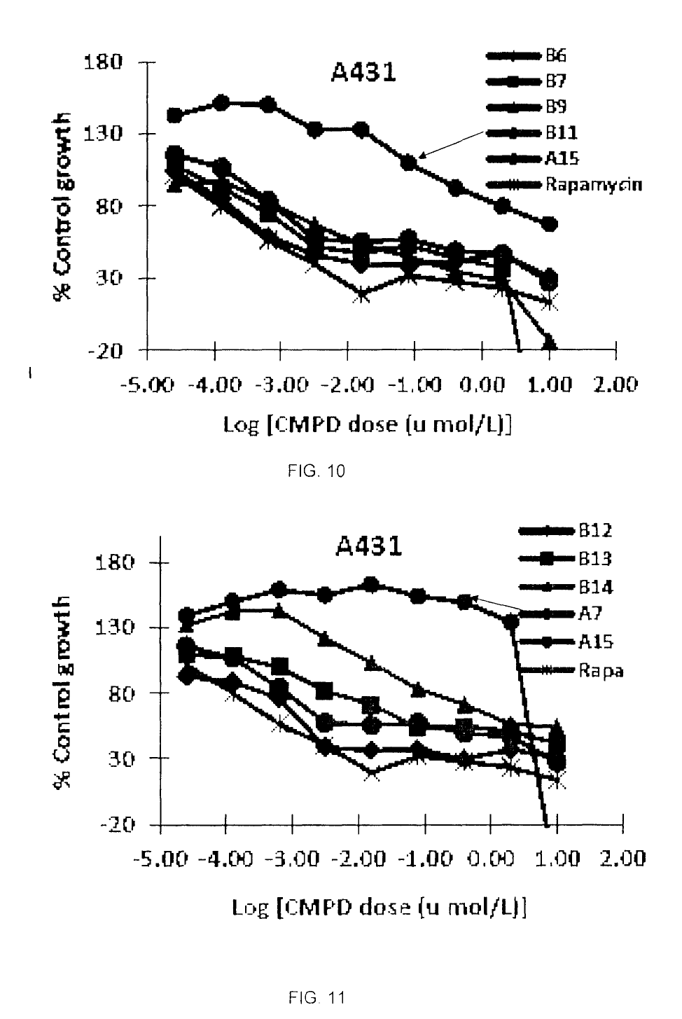

Epidermal cancer A431 tumor cell model: FIG. 9, FIG. 10 and FIG. 11.

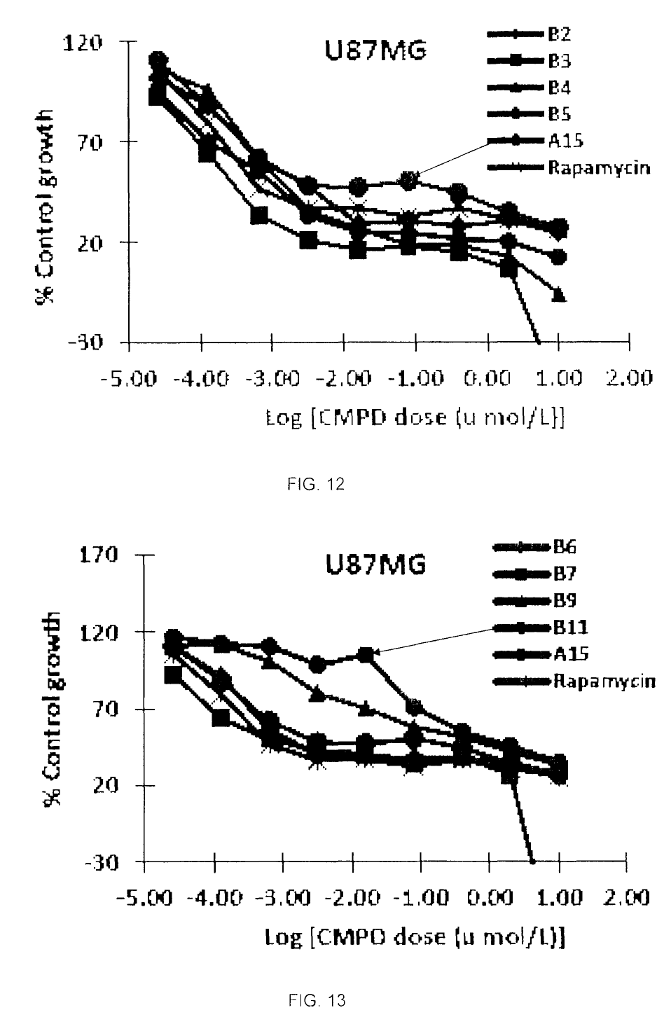

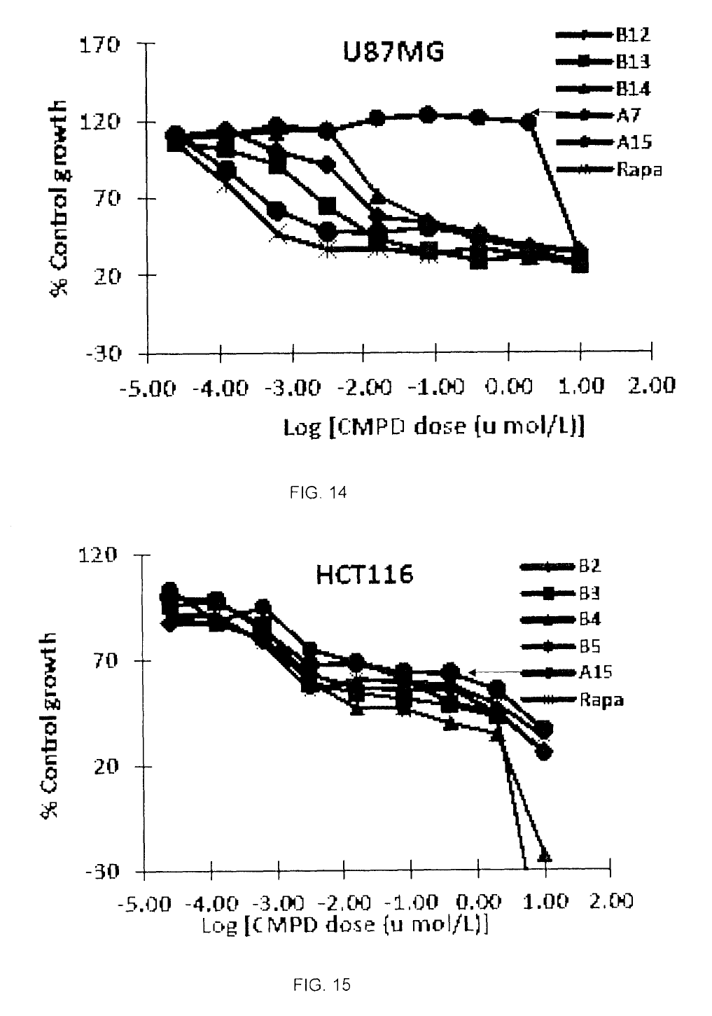

Glioblastoma U87 MG Tumor model studies: FIG. 12, FIG. 13 and FIG. 14.

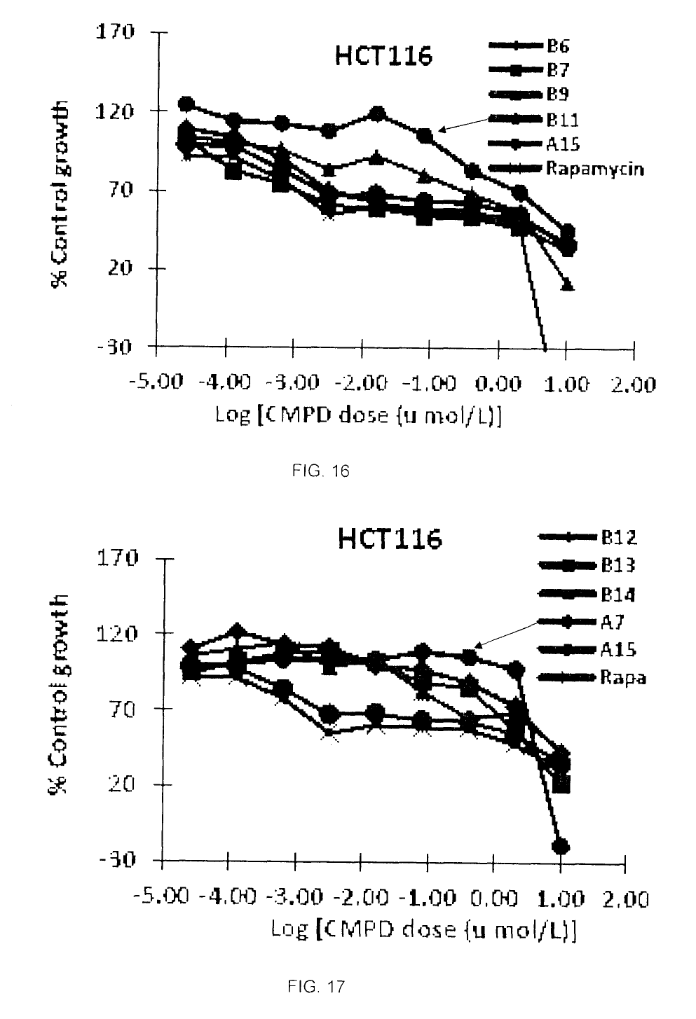

Human colorectal tumor HCT 116 model studies: FIG. 15, FIG. 16 and FIG. 17.

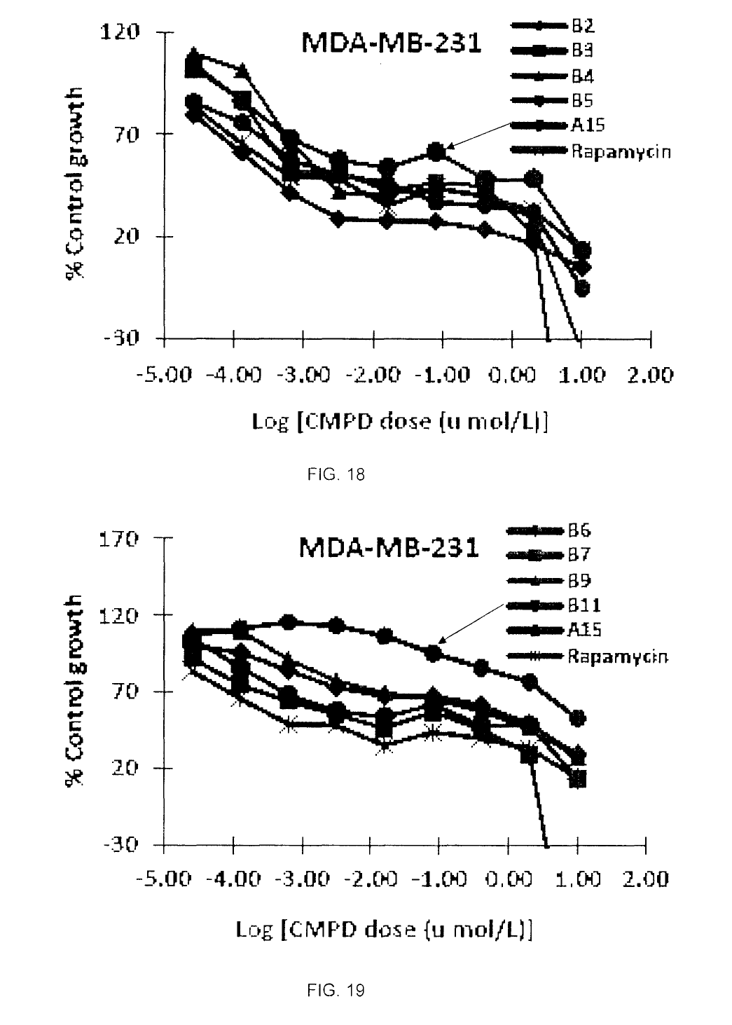

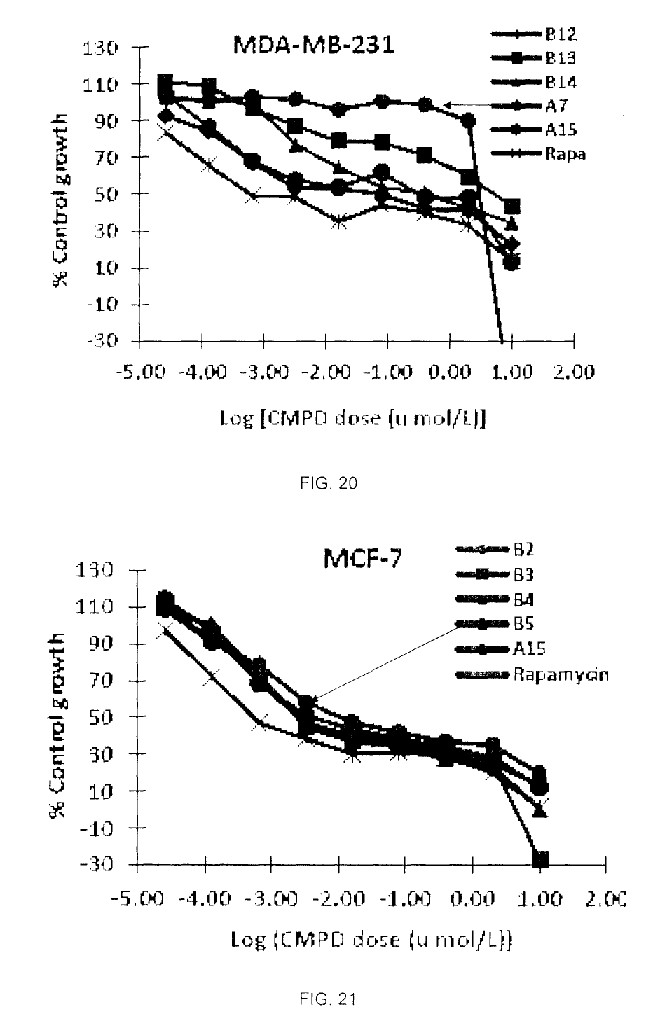

Breast cancer MDA-MB-231 tumor model: FIG. 18, FIG. 19 and FIG. 20.

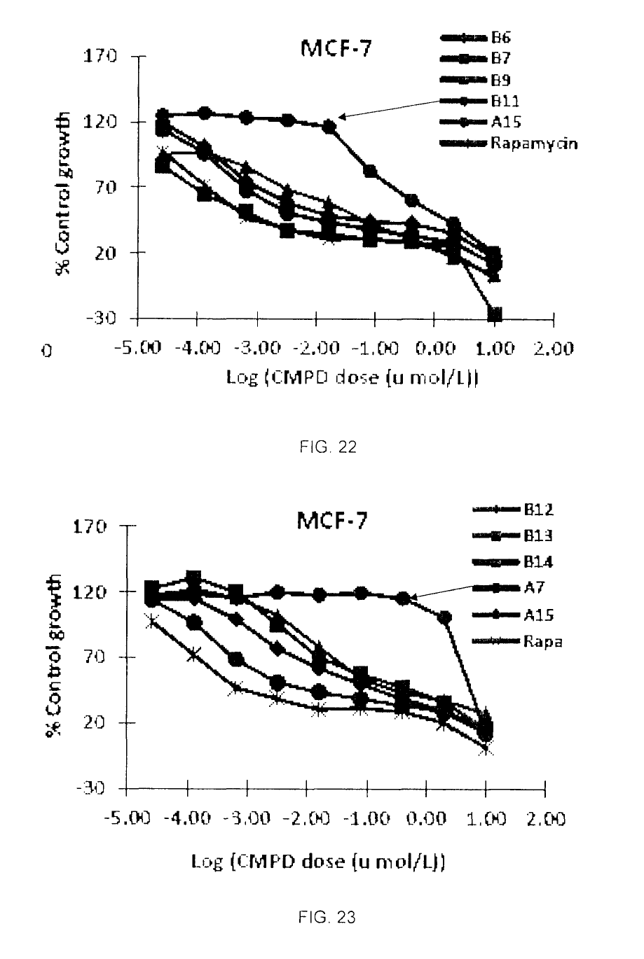

Breast cancer MCF-7 tumor model: FIG. 21, FIG. 22 and FIG. 23.

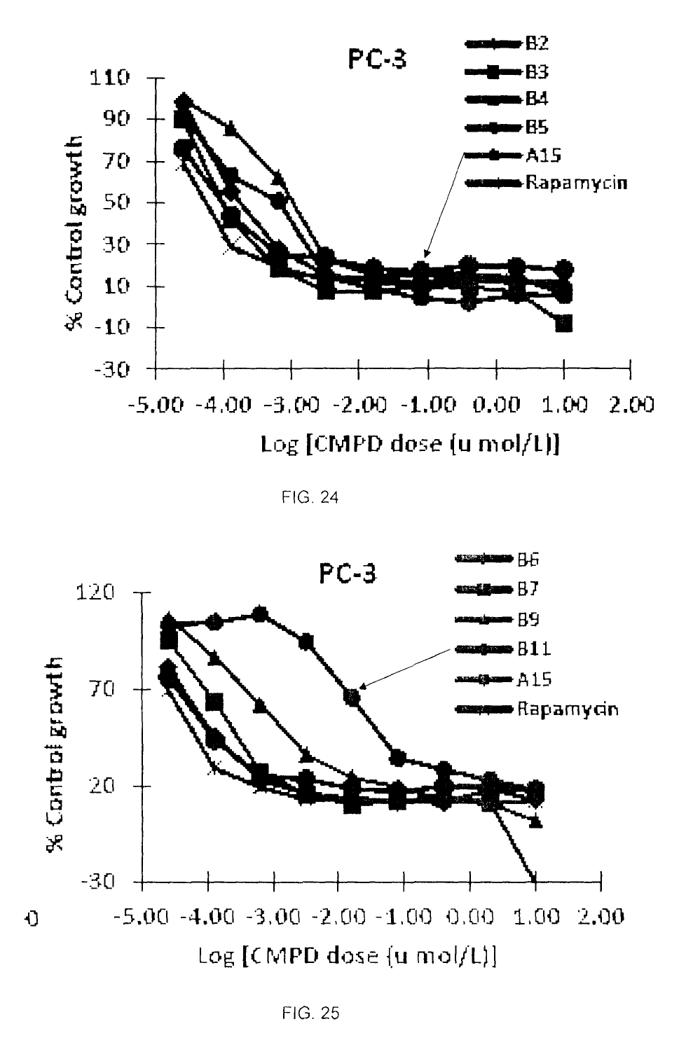

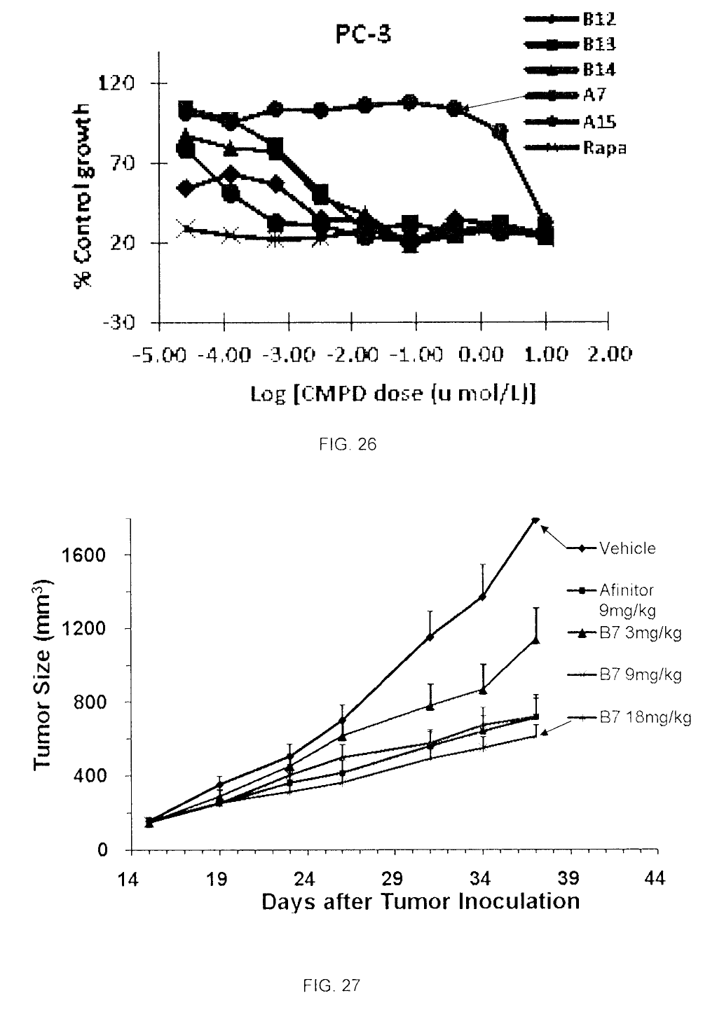

Prostate cancer PC-3 tumor studies: FIG. 24, FIG. 25 and FIG. 26.

### Efficacy Studies of Rapamycin Derivatives in Human Colon Tumor (HCT116) Model

Purpose: The objective of this study is to evaluate preclinically the in vivo therapeutic efficacy of A15 (positive control) and a lead compound from the B series administrated as per os (p.o.) in the slowing or eliminating tumor development in subcutaneous HCT-116 human colon cancer model.

Animals: Balb/c nude mice, female, 6-8 weeks, weighing approximately 18-20 g. A total of 70 will be needed for the study, which will be purchased from Vital River Laboratory Animal Technology Co. Ltd.

Tumor Inoculation: Each mouse will be inoculated subculaneously at the right flank with HCT-116 tumor cells (3×106) in 0.1 ml of PBS for tumor development. The treatments will be started when the tumor size reaches approximately ˜150 mm3. The test article administration and the animal numbers in each group are shown in the following experiment design table.

### Groups and Treatments

| | | | Dose | Dosing | Dosing | |
|---|---|---|---|---|---|---|
| Group | n | Treatment | (mg/kg) | Route | volume | Schedule |
| 1 | 10 | Vehicle | — | p.o. | 10 μl/g | QD × 21 |
| 2 | 10 | A15 | 9 | p.o. | 10 μl/g | QD × 21 |
| 3 | 10 | B | 3 | p.o. | 10 μl/g | QD × 21 |
| 4 | 10 | B | 9 | p.o. | 10 μl/g | QD × 21 |
| 5 | 10 | B | 18 | p.o. | 10 μl/g | QD × 21 |

Note:
n: animal number:
Dosing volume: adjust dosing volume based on body weight 10 μl/g). Treatment schedule may be adjusted if body weight loss >15%.

Assignment to Groups: Before commencement of treatment, all animals will be weighed and the tumor volumes will be measured. Since the tumor volume can affect the effectiveness of any given treatment, mice will be assigned into groups using randomized block design based upon their tumor volumes. This ensures that all the groups are comparable at the baseline.

Endpoints: The major endpoint is to see if the tumor growth can be delayed or mice can be cured. Tumor sizes will be measured twice weekly in two dimensions using a caliper, and the volume will be expressed in mm3 using the formula: V=0.5 a×b2 where a and b are the long and short diameters of the tumor, respectively. The tumor sizes are then used for the calculations of both T-C and T/C values. T-C is calculated with T as the median time (in days) required for the treatment group tumors to reach a predetermined size (e.g., 500 mm3), and C is the median time (in days) for the control group tumors to reach the same size. The T/C value (in percent) is an indication of antitumor effectiveness. T and C are the mean volume of the treated and control groups, respectively, on a given day. Tumor tissues will be collected for the tumor weight and photo at the end of the study.

Termination: This study will be terminated when the mean tumor size of the control group reach the volume of 600-1000 mm3. Animals that are observed to be in a continuing deteriorating condition will be euthanized prior to death, or before reaching a comatose state. Animals showing obvious signs of severe distress and/or pain should be humanely sacrificed. In case of following situations, the animals will be euthanized:

Animals have lost significant body mass (emaciated). Obvious body weight loss>20%.

Animals cannot get to adequate food or water.

The study will be terminated with all animals in all groups being sacrificed when the mean tumor burden in the vehicle treated control group reaches a value of 2000 mm3.

Statistical Analysis: for comparison between two groups, an independent sample t-test will be used. For comparison among three or more groups, a one-way ANOVA will be performed. If a significant F-statistics (a ratio of treatment variance to the error variance) is obtained, multiple comparison procedures will be applied after ANOVA. The potential synergistic effect between treatments will be analyzed by LSD or Dunnett's T3. All data will be analyzed using SPSS 17.0 software, p<0.05 is considered to be statistically significant.

Summary: As shown in the following figure, after 22 days of treatment, the 9 mg/kg/day of A15 positive control compound (Aflinitor from Novartis) significantly inhibited tumor growth by 63% in SubQ HCT116 resistant colon cancer xenograft model(*P<0.05), which is consistent with the reports from literature. After 22 days treatment, B compound suppressed the tumor growth by 42%, 57% and 64% (**, P<0.01), at 3, 9, and 18 mg/kg/day, respectively, compared to the vehicle control. No obvious toxicity was observed. These data indicate that B compound is ery potent in vivo and may overcome the colon cancer resistance.

FIG. 27. In this figure, the top line (diamond) is for Vehicle, second line (triangle) is for B7 at 3 mg/kg dose: third line (purple cross) represents B7 at 9 gm/Kg dose: fourth line from the top (pink square9 represent Afinitor at 9 mg/Kg dose), the bottom line (blue cross) represents B7 a 18 mg/Kg dose.

### Methods of Treatment

The compounds of the present invention, including but not limited to those specified in the examples, possess immunomodulatory and anti-tumor activity in mammals (especially humans). As immunosuppressants, the compounds of the present invention are useful for the treatment and prevention of immune-mediated diseases such as the resistance by transplantation of organs or tissue such as heart, kidney, liver, medulla ossium, skin, cornea, lung, pancreas, intestinum tenue, limb, muscle, nerves, duodenum, small-bowel, pancreatic-islet-cell, and the like: graft-versus-host diseases brought about by medulla ossium transplantation: autoimmune diseases such as rheumatoid arthritis, systemic lupus erythematosus, Hashimoto's thyroiditis, multiple sclerosis, myasthenia gravis, type I diabetes, uveitis, allergic encephalomyelitis, glomerulonephritis, and the like. Further uses include the treatment and prophylaxis of inflammatory and hyperproliferative skin diseases and cutaneous manifestations of immunologically-mediated illnesses, such as psoriasis, atopic dermatitis, contact dermatitis and further eczematous dermatitises, is seborrhoeis dermatitis, lichen planus, pemphigus, bulious pemphigoid, epidermolysis buliosa, urticaria, angioedemas, vasculitides, erythemas, cutaneous eosinophijias, lupus erythematosus, acne and alopecia areata: various eye diseases (autoimmune and otherwise) such as keratoconjunctivitis, vernal conjunctivitis, uveitis associated with Behcet's disease, keratitis, herpetic keratitis, conical cornea, dystrophia epithelialis corneae, corneal leukoma, and ocular pemphigus. In addition, reversible obstructive airway disease, which includes conditions such as asthma (for example, bronchial asthma, allergic asthma, intrinsic asthma, extrinsic asthma and dust asthma), particularly chronic or inveterate asthma (for example, late asthma and airway hyper-responsieness), bronchitis, allergic rhinitis, and the like are targeted by compounds of the present invention. Inflammation of mucosa and blood vessels such as gastric ulcers, vascular damage caused by ischemic diseases and thrombosis. Moreover, hyperproliferative vascular diseases such as intimal smooth muscle cell hyperplasia, restenosis and vascular occlusion, particularly following biologically- or mechanically-mediated vascular injury, may be treated or prevented by the compounds of the present invention. Other treatable conditions include ischemic bowel diseases, inflammatory bowel diseases, necrotizing enterocolitis, intestinal inflammations/allergies such as Coeliac diseases, proctitis, eosinophilic gastroenteritis, mastocytosis, Crohn's disease and ulcerative colitis: nervous diseases such as multiple myositis, Guillain-Barre syndrome, Meniere's disease, polyneuritis, multiple neuritis, mononeuritis and radiculopathy: endocrine diseases such as hyperthyroidism and Basedow's disease: hematic diseases such as pure red cell aplasia, aplastic anemia, hypoplastic anemia, idiopathic thrombocytopenic purpura, autoimmune hemolytic anemia, agranulocytosis, pernicious anemia, megaloblastic anemia and anerythroplasia: bone diseases such as osteoporosis: respiratory diseases such as sarcoidosis, fibroid lung and idiopathic interstitial pneumonia: skin disease such as dermalomyositi, leukoderma vulgaris, ichthyosis vulgaris, photoallergic sensitivity and cutaneous T cell lymphoma: circulatory diseases such as arteriosclerosis, atherosclerosis, aortitis syndrome, polyarteritis nodosa and myocardosis: collagen diseases such as scleroderma, Wegener's granuloma and Sjogren's syndrome: adiposis: eosinophilic fasciitis: periodontal disease such as lesions of gingiva, periodontium, alveolar bone and substantia ossea dentis: nephrotic syndrome such as glomerulonephritis: male pattern aleopecia or alopecia senilis by preventing epilation or providing hair germination and/or promoting hair generation and hair growth: muscular dystrophy: Pyoderma and Sezary's syndrome: Addison's disease: active oxygen-mediated diseases, as for example organ injury such as ischemia-reperfusion injury of organs (such as heart, liver, kidney and digestive tract) which occurs upon preservation, transplantation or ischemic disease (for example, thrombosis and cardiac infarction): intestinal diseases such as endotoxin-shock, pseudomembranous colitis and colitis caused by drug or radiation: renal diseases such as ischemic acute renal insufficiency and chronic renal insufficiency: pulmonary diseases such as toxinosis caused by lung-oxygen or drug (for example, paracort and bleomycins), lung cancer and pulmonary emphysema: ocular diseases such as cataracta, sidcrosis, retinitis, pigmentosa, senile macular degeneration, vitreal scarring and corneal alkali burn: dermatitis such as erythema multiforme, linear IgA ballous dermatitis and cement dermatitis: and others such as gingivitis, periodontitis, sepsis, pancreatitis, diseases caused b_ environmental pollution (for example, air pollution), aging, carcinogenesis, metastasis of carcinoma and hypobaropalhy: diseases caused by histamine or lcukotricne-C.sub.4 release: Behcet's disease such as intestinal-, vasculo- or neuro-Behcet's disease, and also Behcet's which affects the oral cavity, skin, eye, vulva, articulation, epididymis, lung, kidney and so on. Furthermore, the compounds of the present invention may be useful for the treatment and prevention of hepatic disease such as immunogenic diseases (for example, chronic autoimmune liver diseases such as autoimmnune hepatitis, primary biliary cirrhosis and sclerosing cholangitis), partial liver resection, acute liver necrosis (e.g. necrosis caused by toxin, viral hepatitis, shock or anoxia), B-virus hepatitis, non-A/non-B hepatitis, cirrhosis (such as alcoholic cirrhosis) and hepatic failure such as fulminant hepatic failure, late-onset hepatic failure and “acute-on-chronic” liver failure (acute liver failure on chronic liver diseases), and moreover are useful for various diseases because of their useful activity such as augmention of chemotherapeutic effect, cytomegalovirus infection, particularly HCMV infection, anti-inflammatory activity, sclerosing and fibrotic diseases such as nephrosis, scleroderma, pulmonary fibrosis, arteriosclerosis, congestive heart Failure, ventricular hypertrophy, post-surgical adhesions and scarring, stroke, myocardial infarction and injury associated with ischemia and reperfusion, and the like.

Additionally, compounds of the present invention possess FK-506 antagonistic properties. The compounds of the present invention may thus be used in the treatment of immunodepression or a disorder involving immunodepression. Examples of disorders involving immunodepression include AIDS, cancer, fungal infections, senile dementia, trauma (including wound healing, surgery and shock) chronic bacterial infection, and certain central nervous system disorders. The immunodepression to be treated may be caused by an overdose of an immunosuppressive macrocyclic compound, for example derivatives of 12-(2-cyclohcxyl-1-methylvinyl)-13.19.2 1.27-tetramethyl-11.28-dioxa4-azatricyclo[22.3.1.0.sup.4.9]octacos-18-ene such as FK-506 or rapamycin. The overdosing of such medicants by patients is quite common upon their realizing that they have forgotten to take their medication at the prescribed time and may lead to serious side effects.

The compounds of the present invention, including but not limited to those specified in the examples, possess anti-tumor activity in mammals (especially humans). As an anti-cancer drug, the compounds of the invention can be used to treat brain and neurovascular tumors, head and neck cancers, breast cancer, lung cancer, mesothelioma, lymphoid cancer, stomach cancer, kidney cancer, renal carcinoma, liver cancer and liver cirrhosis, ovarian cancer, ovary endometriosis, testicular cancer, skin cancer, melanoma, neuro and all endocrine cancers, spleen cancers, pancreatic cancers, blood proliferative disorders such as Hodgkin's cancer, lymphoma, leukemia, and any cancer disorders that result from uncontrolled cellular proliferations.

The compounds of the present invention, may be mixed with commonly known pharmaceutical exeipicnts such as Eudragit, sodium carboxymelhylcellulose (Na CM), sodium carboxypropyicellulose, any other naturally derived or synthetic exeipicnts to effect an efficacious pharmaceutical formulation. The formulation comprising the compounds of the invention may be made as a immediate release formulation, or a sustained release formulation, or site injection depot formulation, depending on the medical needs. The compound of the present invention may also be combined with a medical device, such as a stent, a balloon, a balloon catheter, an orthopedic device, to further enhance the efficacy of the medical device. The compound of the present invention may be the main function component of a medical treatment regime, such as a local injection formulation, or an ancillary function, such as a coating on a medical device, or in combination with a low-molecular weight or polymer excipient, and used as a coating or filler of a medical device.

When used to treat restenosis following a balloon angioplasty or stent placement, the compounds of the present invention, and the native rapamycin, are thought to exhibit their therapeutic functions through the inhibition of the mammalian target of rapamycin or mTOR. They may also bind to FKBP receptors.

When used in the above or other treatments, a therapeutically effective amount of one of the compounds of the present invention may be employed in pure form or, where such forms exist, in pharmaceutically acceptable salt, ester or prodrug form. Alternately, the compound may be administered as a pharmaceutical composition containing the compound of interest in combination with one or more pharmaceutically acceptable excipients. The phrase “therapeutically effective amount” of the compound of the present invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder: activity of the specilie compound employed: the specific composition employed: the age, body weight, general health, sex and diet of the patient: the time of administration, route of administration, and rate of excretion of the specific compound employed: the duration of the treatment: drugs used in combination or coincidental with the specific compound employed: and like factors well known in the medical arts. For example, it is well within the skill of the art to start doses of the compound at levels lower than required to achieve the desired therapeutic effect and to gradually increase the dosage until the desired effect is achieved.

The total daily dose of the compounds of the present invention administered to a human or lower mammal may range from about 0.01 to about 20 mg/kg/day. For purposes of oral administration, more preferable doses may be in the range of from about 0.001 to about 3 mg/kg/day. If desired, the effective daily dose may be divided into multiple doses for purposes of administration: consequenty, single dose compositions may contain such amounts or submulliples thereof to make up the daily dose. Topical administration may involve doses ranging from 0.001 to 10 percent mg/kg/day, depending on the site of application. When administered locally to treat restenosis and vulnerable plaque, the dose may range from about 1 microgram/mm stent length to about 100 microgram/mm stent length.

### Pharmaceutical Compositions

The pharmaceutical compositions of the present invention comprise a compound and a pharmaceutically acceptable carrier or excipient, which may be administered orally, rectally, parenterally, intracisternally, intravaginally, intraperitonealry, topically (as by powders, ointments, drops or transdermal patch), bucally, or as an oral or nasal spray. The phrase “pharmaceutically acceptable carrier” means a non-toxic solid, semi-solid or liquid filler, diluent, encapsulating material or formulation auxiliary of any type. The term “parenteral,” as used herein, refers to modes of administration which include intravenous, intramuscular, intraperitoneal, intrasternal, subcutaneous and intraarticular injection and infusion.

Pharmaceutical compositions of the present invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternately, delayed absorption of a parenterally administered drug form is accomplished by dissolving or suspending the drug in an oil vehicle.

Injectable depot forms are made by forming microeneapsule matrices of the drug in biodegradable polymers such as polylactide-polyglycolide. Depending upon the ratio of drug to polymer and the nature of the particular polymer employed, the rate of drug release may be controlled. Examples of other biodegradable polymers include poly(orthoesters) and poly(anhydrides). Depot injectable formulations are also prepared by entrapping the drug in liposomes or microemulsions which arc compatible with body tissues.

The injectable formulations may be sterilized, for example, by filtration through a bacterial-retaining filter, or by incorporating sterilizing agents in the form of sterile solid compositions which may be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as carboxy methylcellulose, alginates, gelatin, polyvinylpyrrolidone, sucrose, and acacia, c) humeetants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as cetyl alcohol and glycerol monoslearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft, semi-solid and hard-filled gelatin capsules or liquid-filled capsules using such exeipienls as lactose or milk sugar as well as high molecular weight polyethylene glycols and the like.

The solid dosage forms of tablets, dragees, capsules, pills, and granules may be prepared with coatings and shells such as enteric coatings and other coatings well known in the pharmaceutical formulating art. They may optionally contain opacifying agents and may also be of a composition that they release the active ingredient(s) only, or preferentially, in a certain part of the intestinal tract, optionally, in a delayed manner. Examples of embedding compositions which may be used include polymeric substances and waxes.

The active compounds may also be in a micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art, for example, water or other solvents, solubilizing agents and emulsillers such as ethyl alcohol, isopropvl alcohol, ethyl carbonate, ethyl acetate, benzyl alcohol, benzyl benzoale, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions may also include adjuvants such as wetting agents, emulsifying and suspending agents, sweetening, flavoring, and perfuming agents.

Suspensions, in addition to the active compounds, may contain suspending agents, for example, ethoxy lated isostearyl alcohols, polyoxy ethylene sorbitol and sorbitan esters, microcrystalline cellulose, aluminum metahydroxide, bentonite, agar-agar, and tragacanth, and mixtures thereof.

Topical administration includes administration to the skin or mucosa, including surfaces of the lung and eye. Compositions for topical administration, including those for inhalation, may be prepared as a dry powder which may be pressurized or non-pressurized. In non-pressurized powder compositions, the active ingredient in finely divided form may be used in admixture with a larger-sized pharmaceutically acceptable inert carrier comprising particles having a size, for example, of up to 100 micrometers in diameter. Suitable inert carriers include sugars such as lactose. Desirably, at least 95 percent by weight of the particles of the active ingredient have an effective particle size in the range of 0.01 to 10 micrometers. Compositions for topical use on the skin also include ointments, creams, lotions, and gels.

Alternately, the composition may be pressurized and contain a compressed gas, such as nitrogen or a liquefied gas propellant. The liquefied propellanl medium and indeed the total composition is preferably such that the active ingredient does not dissolve therein to any substantial extent. The pressurized composition may also contain a surface active agent. The surface active agent may be a liquid or solid non-ionic surface active agent or may be a solid anionic surface active agent. It is preferred to use the solid anionic surface active agent in the form of a sodium salt.

A further form of topical administration is to the eye, as for the treatment of immune-mediated conditions of the eye such as autoimmune diseases, allergic or inflammatory conditions, and corneal transplants. The compound of the present invention is delivered in a pharmaceutically acceptable ophthalmic vehicle, such that the compound is maintained in contact with the ocular surface for a sufficient time period to allow the compound to penetrate the corneal and internal regions of the eye, as for example the anterior chamber, posterior chamber, vitreous body, aqueous humor, vitreous humor, cornea, iris/cilary, lens, choroid/retina and sclera. The pharmaceutically acceptable ophthalmic vehicle may, for example, be an ointment, vegetable oil or an encapsulating material.

Compositions for rectal or vaginal administration are preferably suppositories or retention enemas which may be prepared by mixing the compounds of the present invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention may also be administered in the form of liposomes. As is known in the art, liposomes arc generally derived from phospholipids or other lipid substances, liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form may contain, in addition to a compound of the present invention, stabilizers, preservatives, excipients, and the like. The preferred lipids are the phospholipids and the phosphatidyl cholines (lecithins), both natural and synthetic. Methods to form liposomes arc known in the art. See, for example, Prescott, Ed., Methods in Cell Biology, Volume XIV, Academic Press, New York, N.Y. (1976), p. 33 et seq.

Compounds of the present invention may also be coadministered with one or more immunosuppressant agents. The immunosuppressant agents within the scope of the present invention include IMURAN® azathioprine sodium, brequinar sodium, SPANIDIN® guspcrimus trihydrochloride (also known as dcoxyspergualin), mizoribine (also known as bredinin), CELLCEPT® mycophenolate mofetil, NEORAl®, Cylosporin A (also marketed as different formulation of Cyclosporin A under the trademark SANDIMMUNE®, PROGRAE®, tacrolimus (also known as FK-506), sirolimus and RAPAMUNE®, leflunomide (also known as HWA186), glucocorticoids, such as prednisolone and its derivatives, antibody therapies such as orthoclone (OKT3) and Zenapax®, and antithymyocyte globulins, such as thymoglobulins.

The local delivery of drug/drug combinations from a stent or other implantable device has the following advantages: namely, the prevention of vessel recoil and remodeling through the scaffolding action of the stent and the prevention of multiple components of neointimal hyperplasia or restenosis as well as a reduction in inflammation and thrombosis. This local administration of drugs, agents or compounds to stented coronary arteries may also have additional therapeutic benefit. For example, higher tissue concentrations of the drugs, agents or compounds may be achieved utilizing local delivery, rather than systemic administration. In addition, reduced systemic toxicity may be achieved utilizing local delivery rather than systemic administration while maintaining higher tissue concentrations. Also in utilizing local delivery from a stent rather than systemic administration, a single procedure may suffice with better patient compliance. An additional benefit of combination drug, agent, and/or compound therapy may be to reduce the dose of each of the therapeutic drugs, agents or compounds, thereby limiting their toxicity, while still achieving a reduction in restenosis, inflammation and thrombosis. Local stent-based therapy is therefore a means of improving the therapeutic ratio (efficacy toxicity) of anti-restenosis, anti-inflammatory, antithrombotic drugs, agents or compounds.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

What is claimed is:

1. A compound of Formula I or a pharmaceutically acceptable salt or prodrug thereof: 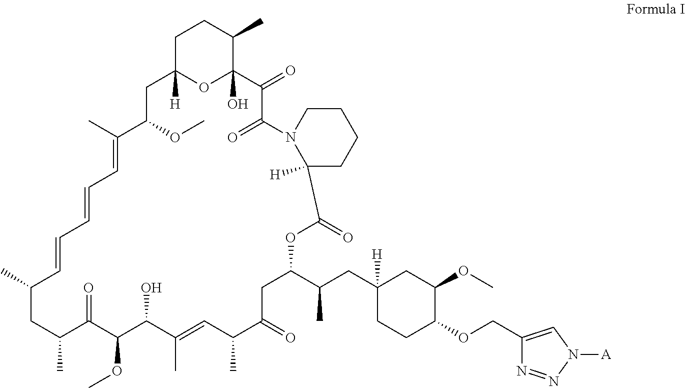
wherein A is selected from the group consisting of:
a) hydrogen, alkyl and substituted alkyl, alkenyl and substituted alkenyl, alkynyl and substituted alkynyl, cycloalkyl and substituted cycloalkyl, heterocycloalkyl and substituted heterocycloalkyl; the substitution group including hydroxyl, sulfonyl, carbonyl, amino, cyano, halogen, alkoxy, aryl, and heteroaryl, and
b) aryl and substituted aryl, heteroaryl and substituted heteroaryl; the substitution group including hydroxyl, halogen, amino, carbonyl, cyano, nitro, sulfonyl, alkyl, alkoxy, cycloalkyl, heterocycloalkyl.

2. The compound from claim 1, wherein the compound is selected from the group consisting of:

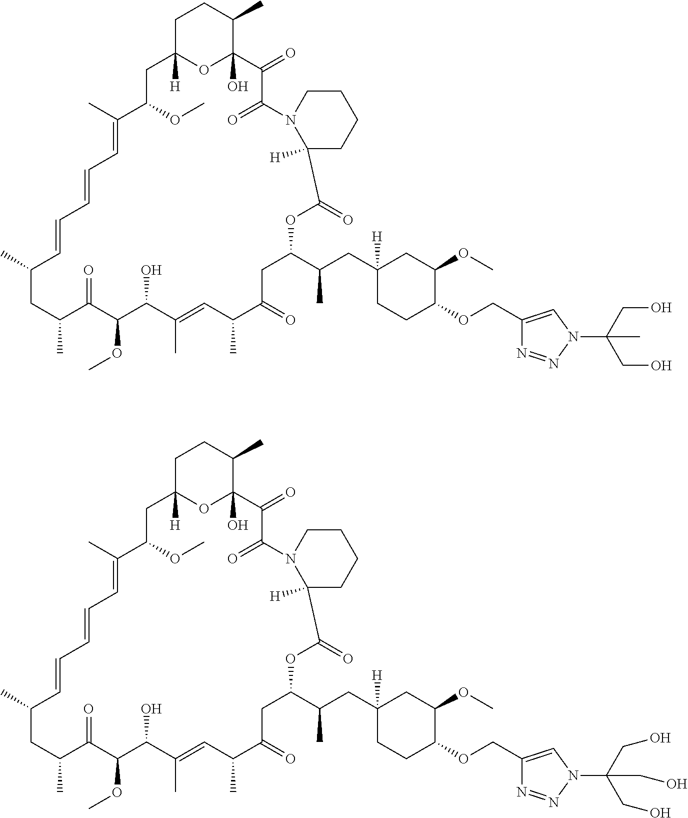 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCc3cn(C(C)(CO)CO)nn3)[C@H](OC)C2)CC(=O)[C@H](C)/C=C(\C)[C@@H](O)[C@@H](OC)C(=O)[C@H](C)C[C@H](C)/C=C/C=C/C=C/1C ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCc3cn(C(CO)(CO)CO)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

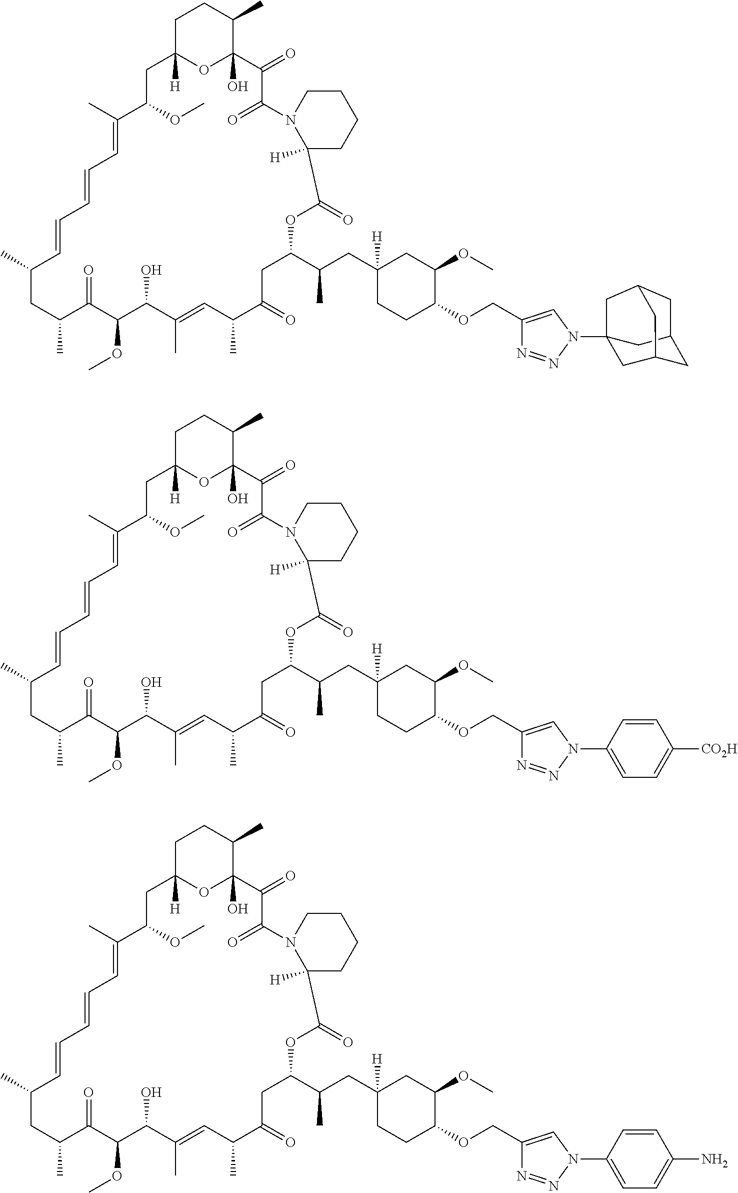

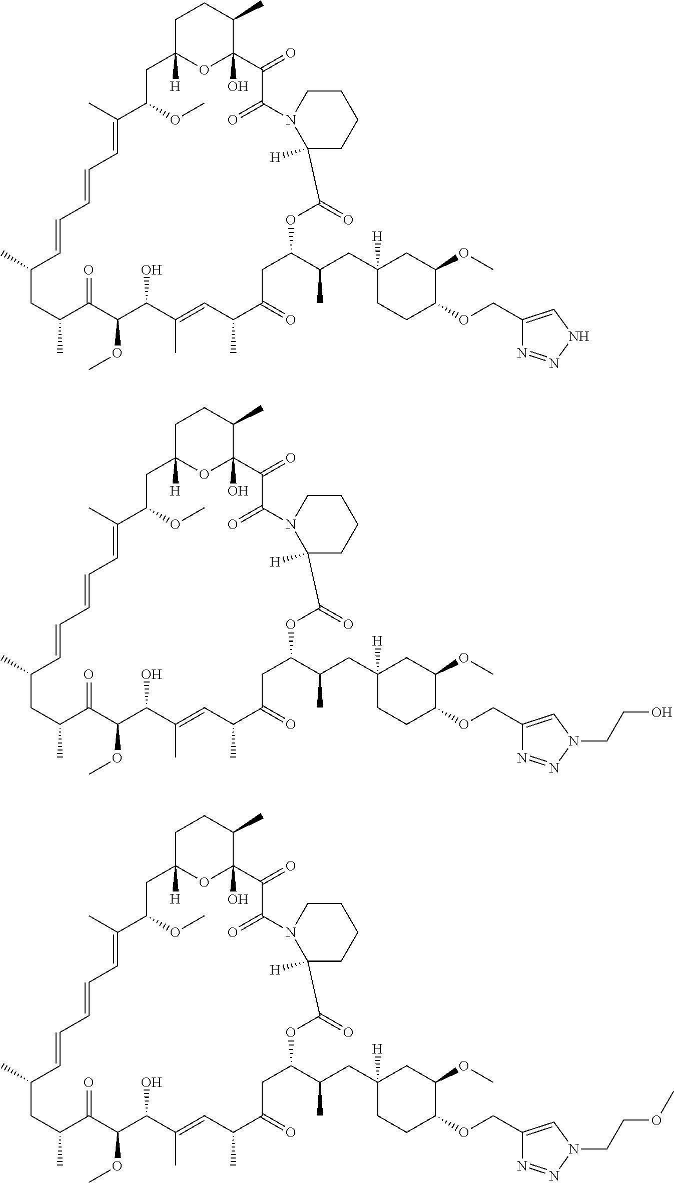

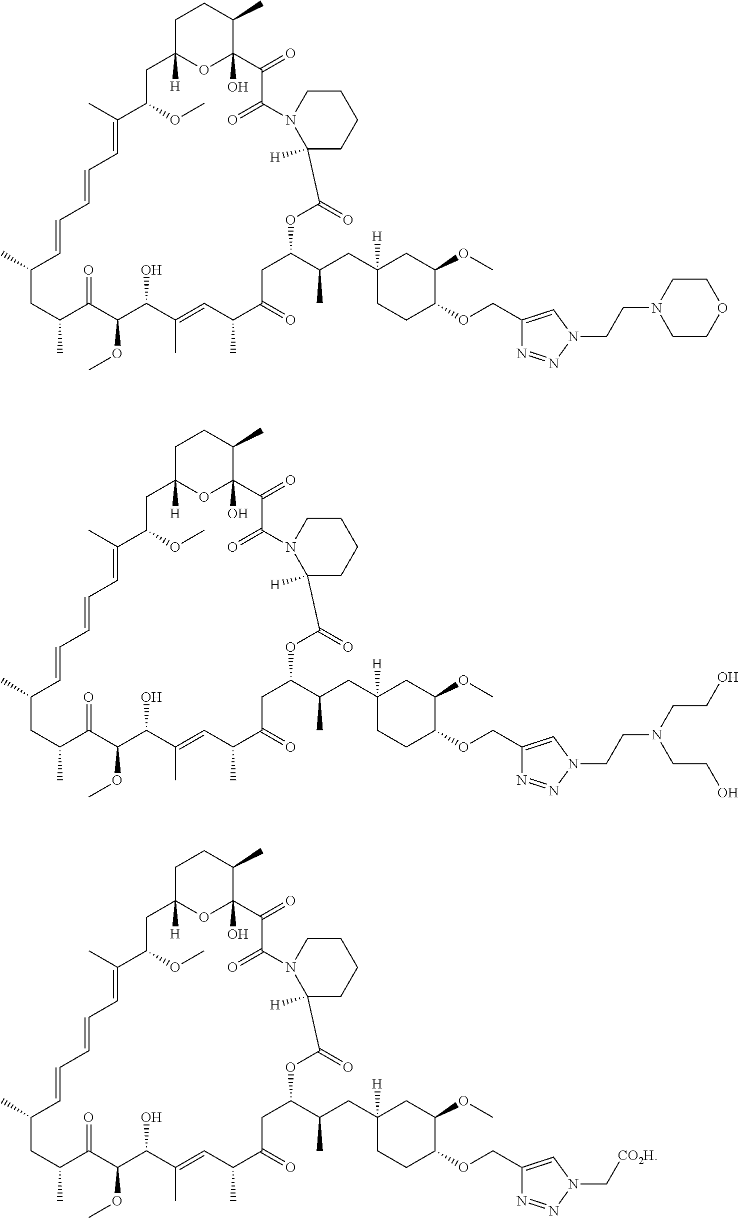

3. A compound of Formula II or a pharmaceutically acceptable salt or prodrug thereof: 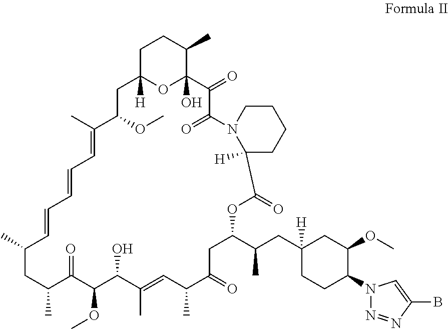
wherein B is selected from the group consisting of:
a) hydrogen, alkyl and substituted alkyl, alkenyl and substituted alkenyl, alkynyl and substituted alkynyl, cycloalkyl and substituted cycloalkyl, heterocycloalkyl and substituted heterocycloalkyl; wherein each substituent is independently hydroxyl, sulfonyl, carbonyl, amino, cyano, halogen, alkoxy, aryl, or heteroaryl, and
b) aryl and substituted aryl, heteroaryl and substituted heteroaryl; wherein each substituent is hydroxyl, halogen, amino, carbonyl,cyano, nitro, sulfonyl, alkyl, alkoxy, cycloalkyl, or heterocycloalkyl.

4. The compound from claim 3, wherein the compound is selected from the group consisting of:

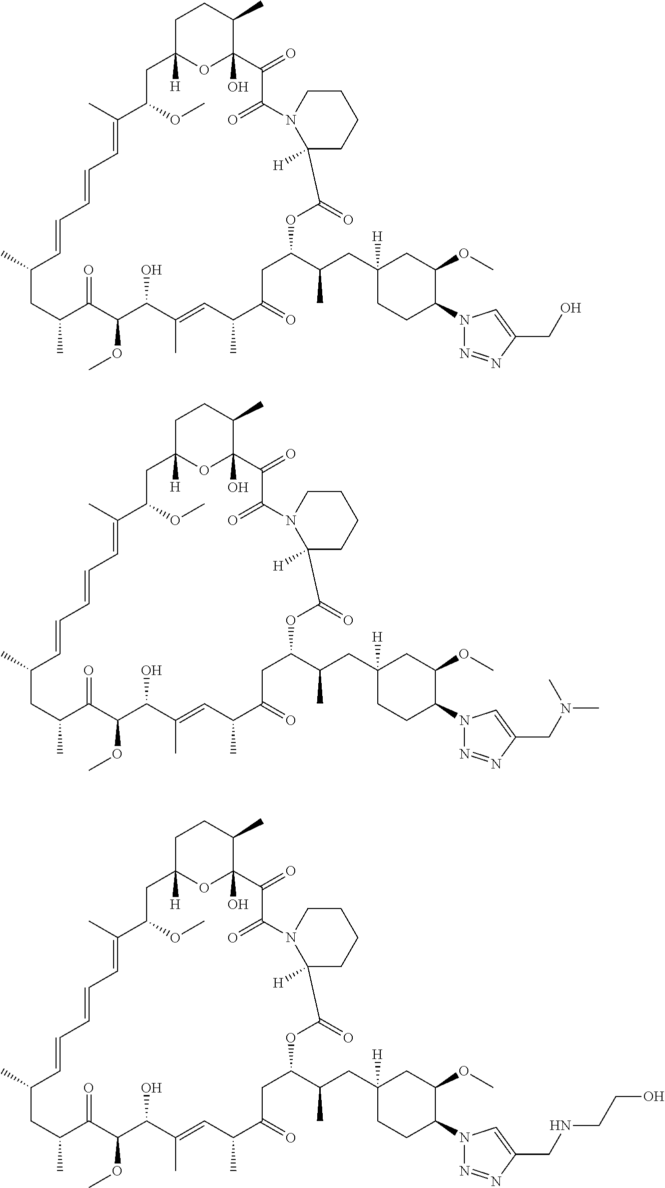

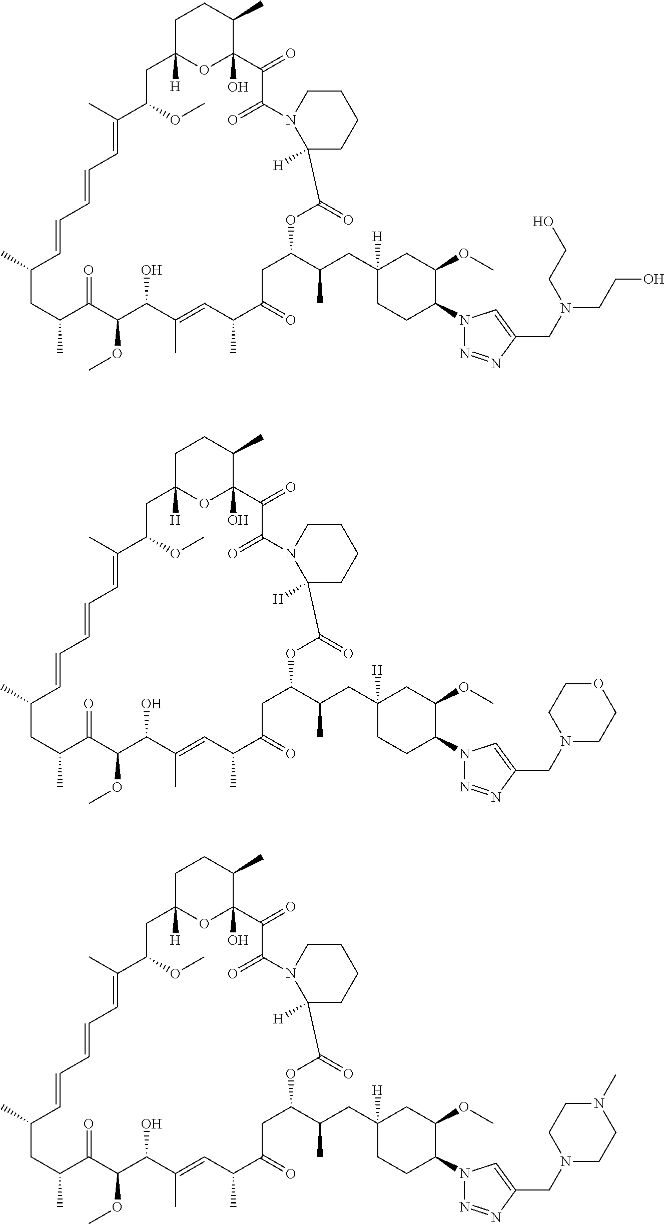

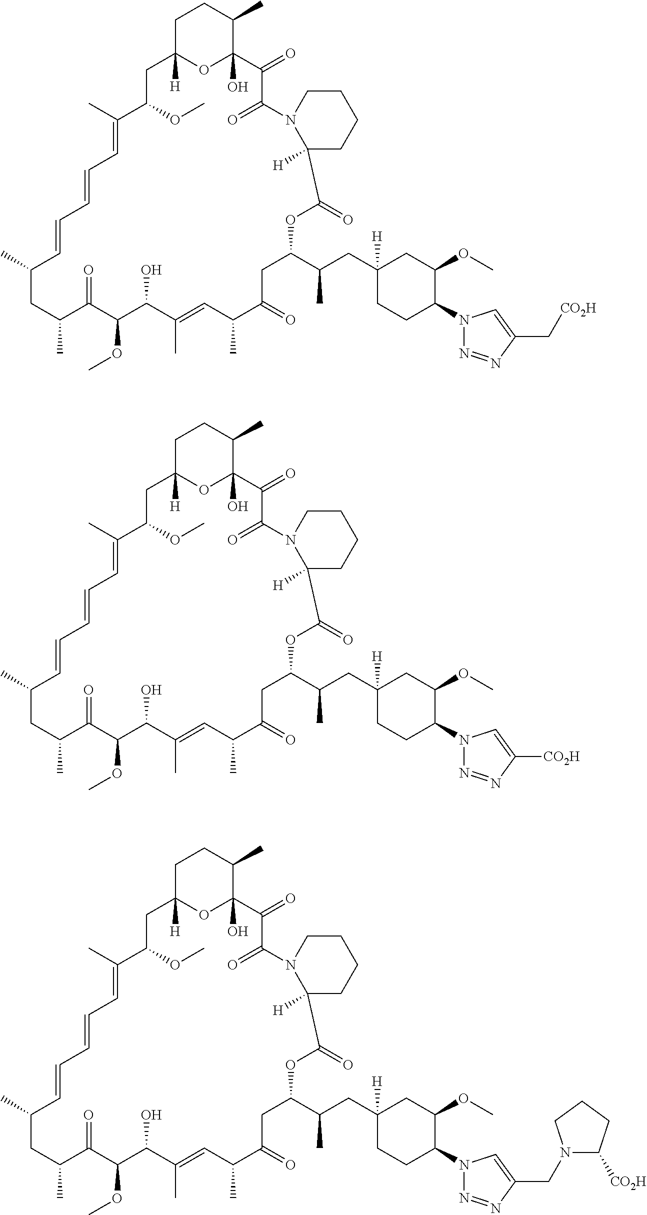

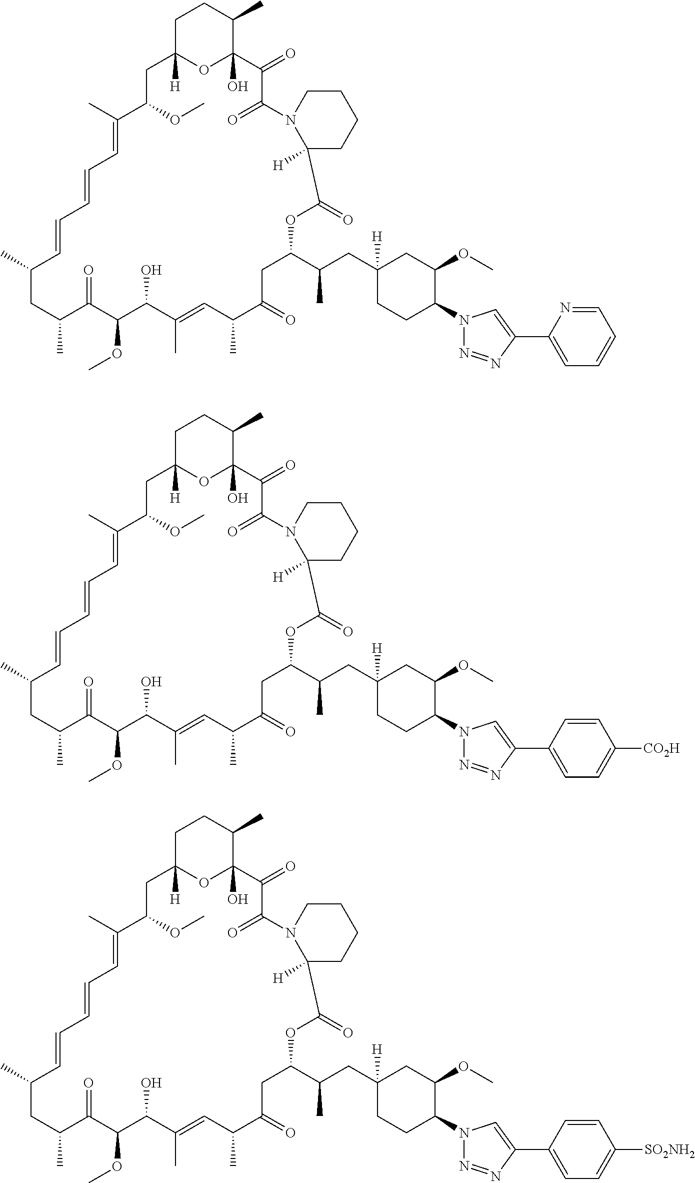

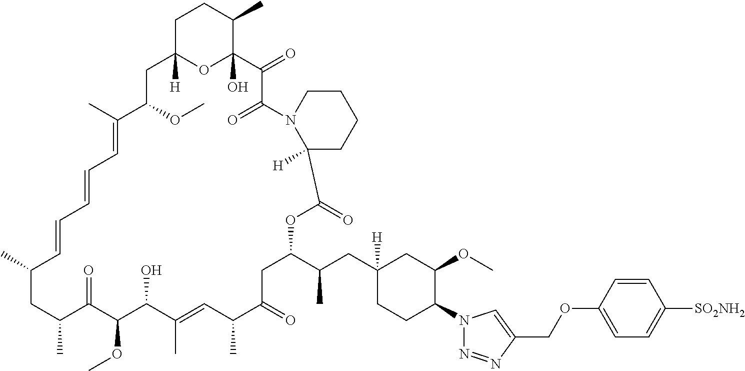 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COc4ccc(S(N)(=O)=O)cc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

5. A pharmaceutical composition comprising a compound of any of claims 1-4 and a pharmaceutical excipient.

6. The pharmaceutical formulation of claim 5, wherein the formulation is suitable for administration to a mammal via a route selected from the group consisting of oral, nasal, intravenous, transdermal, parenteral, subcutaneous, intramuscular, intra-ocular, and peritoneal routes.

7. The pharmaceutical formulation of claim 6, wherein the mammal is a human.

8. A method of treating cancer, comprising administering to a subject in need thereof a therapeutically effective amount of a compound of any of claims 1-4.

9. The method of claim 8, wherein the cancer is selected from the group consisting of brain and neurovascular tumors, head and neck cancers, breast cancer, lung cancer, mesothelioma, lymphoid cancer, stomach cancer, kidney cancer, renal carcinoma, liver cancer and liver cirrhosis, ovarian cancer, ovary endometriosis, testicular cancer, skin cancer, melanoma, neuro and all endocrine cancers, spleen cancers, pancreatic cancers, blood proliferative disorders such as Hodgkin's cancer, lymphoma, leukemia, and any cancer disorders that result from uncontrolled cellular proliferations

10. A method for treating or preventing an immune-mediated disease in a subject, comprising administering to the subject in need thereof a therapeutically effective amount of a compound of any of claims 1-4.

11. The method of claim 10, wherein the immune-mediated disease is selected from the group consisting of resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation.

12. The method of claim 10, wherein the immune-mediated disease is rheumatoid arthritis, systemic lupus erythematosus, Hashimoto's thyroiditis, multiple sclerosis, myasthenia gravis, type I diabetes, uveitis, allergic encephalomyelitis, or glomerulonephritis.

13. The method of claim 10, wherein the immune-mediated disease is a graft-versus-host disease brought about by medulla ossium transplantation.

14. Use of a compound of any of claims 1-4 for manufacturing of a medicament for the treatment of a cancer or an immune-mediated disease.

15. The use of claim 14, wherein the cancer is selected from the group consisting of brain and neurovascular tumors, head and neck cancers, breast cancer, lung cancer, mesothelioma, lymphoid cancer, stomach cancer, kidney cancer, renal carcinoma, liver cancer and liver cirrhosis, ovarian cancer, ovary endometriosis, testicular cancer, skin cancer, melanoma, neuro and all endocrine cancers, spleen cancers, pancreatic cancers, blood proliferative disorders such as Hodgkin's cancer, lymphoma, leukemia, and any cancer disorders that result from uncontrolled cellular proliferations; and the immune-mediated disease is selected from the group consisting of resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation.
